(19) United States
(12) Patent Application Publication
(10) Pub. No.: US 2026/0098044 A1
(43) Pub. Date: Apr. 9, 2026

(54) TRICYCLIC HETEROARYL COMPOUNDS AS INHIBITORS OF TYK2 AND/OR JAK1

(71) Applicant: Incyte Corporation, Wilmington, DE (US)

(72) Inventors: Stacey Shepard, Wilmington, DE (US); Onur Atasoylu, Wilmington, DE (US); Song Mei, Wilmington, DE (US); Jun Pan, Media, PA (US)

(21) Appl. No.: 19/348,390

(22) Filed: Oct. 2, 2025

Related U.S. Application Data
(60) Provisional application No. 63/703,774, filed on Oct. 4, 2024.

(51) Int. Cl. C07D 495/14; A61K 31/437; A61K 31/444; A61K 31/496; A61K 31/497; A61K 31/501; A61K 31/506; A61K 31/5377; C07D 519/00

(52) U.S. Cl. CPC C07D 495/14; A61K 31/437; A61K 31/444; A61K 31/496; A61K 31/497; A61K 31/501; A61K 31/506; A61K 31/5377; C07D 519/00

(57) ABSTRACT

The present application provides tricyclic heteroaryl compounds that modulate the activity of the JAK1 and/or TYK2, which are useful in the treatment of various disorders and diseases, including skin, respiratory, ophthalmic, and rectal diseases and disorders, cancer, and neuroinflammation.

### CLAIM OF PRIORITY

This application claims the benefit of priority of U.S. Prov. Appl. No. 63/703,774, filed Oct. 4, 2024, which is incorporated herein by reference in its entirety.

### FIELD

The present disclosure provides tricyclic heteroaromatic compounds that modulate the activity of TYK2 and/or JAK1 kinases and are useful in the treatment of diseases associated with TYK2 and/or JAK1, including skin, respiratory, ophthalmic, and rectal diseases and disorders, cancer, and neuroinflammation.

### BACKGROUND

Protein kinases (PKs) regulate diverse biological processes including cell growth, survival, differentiation, organ formation, morphogenesis, neovascularization, tissue repair, and regeneration, among others. Protein kinases also play specialized roles in a host of human diseases including cancer. Cytokines, low-molecular weight polypeptides or glycoproteins, regulate many pathways involved in the host inflammatory response to sepsis. Cytokines influence cell differentiation, proliferation and activation, and can modulate both pro-inflammatory and anti-inflammatory responses to allow the host to react appropriately to pathogens. Signaling of a wide range of cytokines involves the Janus kinase family (JAKs) of protein tyrosine kinases and Signal Transducers and Activators of Transcription (STATs). There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Cytokine-stimulated immune and inflammatory responses contribute to pathogenesis of diseases: pathologies such as severe combined immunodeficiency (SCID) arise from suppression of the immune system, while a hyperactive or inappropriate immune/inflammatory response contributes to the pathology of autoimmune diseases (e.g., asthma, systemic lupus erythematosus, thyroiditis, myocarditis), and illnesses such as scleroderma and osteoarthritis (Ortmann, R. A., T. Cheng, et al. (2000) Arthritis Res 2(1): 16-32).

Deficiencies in expression of JAKs are associated with many disease states. For example, Jak1−/− mice are runted at birth, fail to nurse, and die perinatally (Rodig, S. J., M. A. Meraz, et al. (1998) Cell 93(3): 373-83). Jak2−/− mouse embryos are anemic and die around day 12.5 postcoitum due to the absence of definitive erythropoiesis.

The JAK/STAT pathway, and in particular all four JAKs, are believed to play a role in the pathogenesis of asthmatic response, chronic obstructive pulmonary disease, bronchitis, and other related inflammatory diseases of the lower respiratory tract. Multiple cytokines that signal through JAKs have been linked to inflammatory diseases/conditions of the upper respiratory tract, such as those affecting the nose and sinuses (e.g., rhinitis and sinusitis) whether classically allergic reactions or not. The JAK/STAT pathway has also been implicated in inflammatory diseases/conditions of the eye and chronic allergic responses.

Activation of JAK/STAT in cancers may occur by cytokine stimulation (e.g. IL-6 or GM-CSF) or by a reduction in the endogenous suppressors of JAK signaling such as SOCS (suppressor or cytokine signaling) or PIAS (protein inhibitor of activated STAT) (Boudny, V., and Kovarik, J., Neoplasm. 49:349-355, 2002). Activation of STAT signaling, as well as other pathways downstream of JAKs (e.g., Akt), has been correlated with poor prognosis in many cancer types (Bowman, T., et al. Oncogene 19:2474-2488, 2000). Elevated levels of circulating cytokines that signal through JAK/STAT play a causal role in cachexia and/or chronic fatigue. As such, JAK inhibition may be beneficial to cancer patients for reasons that extend beyond potential anti-tumor activity.

JAK1 plays a central role in a number of cytokine and growth factor signaling pathways that, when dysregulated, can result in or contribute to disease states. For example, IL-6 levels are elevated in rheumatoid arthritis, a disease in which it has been suggested to have detrimental effects (Fonesca, J. E. et al., Autoimmunity Reviews, 8:538-42, 2009). Because IL-6 signals, at least in part, through JAK1, antagonizing IL-6 directly or indirectly through JAK1 inhibition is expected to provide clinical benefit (Guschin, D., N., et al Embo J 14:1421, 1995; Smolen, J. S., et al. Lancet 371:987, 2008). Moreover, in some cancers JAK1 is mutated resulting in constitutive undesirable tumor cell growth and survival (Mullighan C G, Proc Natl Acad Sci USA. 106:9414-8, 2009; Flex E., et al. J Exp Med. 205:751-8, 2008). In other autoimmune diseases and cancers elevated systemic levels of inflammatory cytokines that activate JAK1 may also contribute to the disease and/or associated symptoms. Therefore, patients with such diseases may benefit from JAK1 inhibition. Selective inhibitors of JAK1 may be efficacious while avoiding unnecessary and potentially undesirable effects of inhibiting other JAK kinases.

Selective inhibitors of JAK1, relative to other JAK kinases (particularly JAK2 and JAK3), may have multiple therapeutic advantages over less selective inhibitors. With respect to selectivity against JAK2, a number of important cytokines and growth factors signal through JAK2 including, for example, erythropoietin (EPO) and thrombopoietin (Tpo) (Parganas E, et al. Cell. 93:385-95, 1998). EPO is a key growth factor for red blood cells production; hence a paucity of EPO-dependent signaling can result in reduced numbers of red blood cells and anemia (Kaushansky K, NEJM 354:2034-45, 2006). Tpo, another example of a JAK2-dependent growth factor, plays a central role in controlling the proliferation and maturation of megakaryocytes—the cells from which platelets are produced (Kaushansky K, NEJM 354:2034-45, 2006). As such, reduced Tpo signaling would decrease megakaryocyte numbers (megakaryocytopenia) and lower circulating platelet counts (thrombocytopenia). This can result in undesirable and/or uncontrollable bleeding. Reduced inhibition of other JAKs, such as JAK3 and TYK2, may also be desirable as humans lacking functional version of these kinases have been shown to suffer from numerous maladies such as severe-combined immunodeficiency or hyperimmunoglobulin E syndrome (Minegishi, Y, et al. Immunity 25:745-55, 2006; Macchi P, et al. Nature. 377:65-8, 1995). Therefore a JAK1 inhibitor with reduced affinity for other JAKs would have significant advantages over a less-selective inhibitor with respect to reduced side effects involving immune suppression, anemia and thrombocytopenia.

TYK2-deficient mice are resistant in experimental models of colitis, psoriasis, and multiple sclerosis, suggesting an important role for TYK2-mediated signaling in autoimmunity and related diseases. Individuals expressing inactive variants of TYK2 in humans are protected from multiple sclerosis and other autoimmune diseases. Genome-wide association studies have shown that other variants of TYK2 are associated with autoimmune diseases such as Crohn's disease, psoriasis, systemic lupus erythematosus and rheumatoid arthritis, further demonstrating the importance of TYK2 in autoimmunity.

TYK2 knockout mice had normal red blood cell counts and were able to survive. TYK2 deficiency is manifested by impaired signal transduction of various pro-inflammatory cytokines and severe imbalance of T helper cell differentiation. Results from gene association studies support that TYK2 is a common susceptibility gene for autoimmune diseases. TYK2-regulated channels have also been further confirmed by antibody therapy in disease treatment, such as targeting IL-12/IL-23, ustekinumab for the treatment of psoriasis and targeting type I interferon receptors, Anifrolumab for the treatment of systemic lupus erythematosus (SLE) has shown significant efficacy in clinical trials.

TYK2 is also associated with some cancers, such as the abnormal survival of acute lymphoblastic leukemia (T-ALL) cells associated with the activation of TYK2. Gene knockout experiments showed that 88% of T-ALL cell lines and 63% of patient-derived T-ALL cells were TYK2-dependent, thus, TYK2 is an oncogene in T-ALL (Sanda et. al, Cancer Disc. 2013, 3, 564-77). TYK2-selective inhibitor NDI-031301 induces apoptosis to inhibit the growth of human T-ALL cell lines, and has good safety and efficacy in a mouse model with KOPT-K1 T-ALL tumor cells (Akahane et. al, British J. Haematol. 2017, 177, 271-82), showing the promise of TYK2-selective inhibitors in the treatment of T-ALL. Therefore, TYK2 is one of the popular targets for the treatment of inflammatory diseases, autoimmune diseases and cancer (Alicea-Velazquez et. al, Curr. Drug Targets 2011, 12, 546-55).

Thus, new or improved agents which inhibit JAK1 and/or TYK2 are continually needed for developing new and more effective pharmaceuticals that are aimed at augmentation or suppression of the immune and inflammatory pathways (such as immunosuppressive agents for organ transplants), as well as agents for the prevention and treatment of autoimmune diseases, diseases involving a hyperactive inflammatory response (e.g., eczema), allergies, cancer (e.g., prostate, leukemia, multiple myeloma), and some immune reactions (e.g., skin rash or contact dermatitis or diarrhea) caused by other therapeutics. The present disclosure described herein is directed toward this end and others.

### SUMMARY

The present disclosure relates to, inter alia, compounds of Formula I:

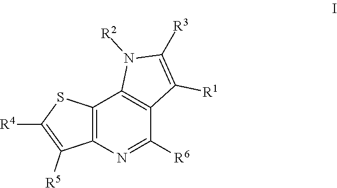

or pharmaceutically acceptable salts thereof, wherein constituent members are defined infra.

In some embodiments, the compounds or the salts thereof are inhibitors of JAK1. In some embodiments, the compounds or the salts thereof are inhibitors of TYK2. In some embodiments, the compounds or the salts thereof are inhibitors of JAK1 and TYK2. In some embodiments, the compounds or the salts thereof are selective inhibitors of JAK1 and/or TYK2. In some embodiments, the compounds or the salts thereof are selective inhibitors of JAK1 and TYK2. In some embodiments, the compounds or the salts thereof are brain penetrants.

The present invention further provides pharmaceutical compositions comprising a compound of Formula I, or a pharmaceutically acceptable salt thereof, and a pharmaceutically acceptable carrier.

The present invention further provides methods of treating a disease or a disorder mediated by TYK2 and/or JAK1 in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I:

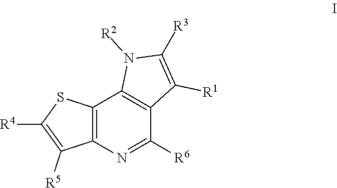

or a pharmaceutically acceptable salt thereof, wherein:
R¹ is -(L¹)-R^{X} or —R^{Y};
L¹ is C₁₋₆ alkylene, C₂₋₆ alkenylene, C₂₋₆ alkynylene, C₁₋₆ haloalkylene, —O—(C₁₋₆ alkylene)-, or —N(R^{L})—, wherein said C₁₋₆ alkylene, C₂₋₆ alkenylene, C₂₋₆ alkynylene, C₁₋₆ haloalkylene, and —O—(C₁₋₆ alkylene)- are each optionally substituted by 1, 2, 3, or 4 independently selected R^{G} substituents;
R^{L} is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
R^{X} is selected from Cy¹, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{1c}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1};
R^{Y} is selected from Cy¹, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1};
Cy¹ is C₃₋₁₀ cycloalkyl, 6-10 membered aryl, 4-10 membered heterocycloalkyl, or 5-10 membered heteroaryl, each of which is optionally substituted with 1, 2, 3, 4, 5, or 6 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂-6 alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or two R^{1A} substituents attached to C₃₋₁₀ cycloalkyl, 6-10 membered aryl, 4-10 membered heterocycloalkyl, or 5-10 membered heteroaryl, together with the atoms to which they are connected, form a C₃₋₇ cycloalkyl, phenyl, 4-7 membered heterocycloalkyl, or 5-6 membered heteroaryl ring, which is optionally substituted by 1, 2, 3, or 4 independently selected R^{Cy1} substituents;
each R^{Cy1} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b1}, NR^{c11}S(O)₂R^{b1}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12} and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
or, any R^{c12} and R^{d12} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{1C} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1C} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, NHOR^{a13}, C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13}, NR^{c13}R^{d13}, NR^{c13}NR^{c13}R^{d13}, NR^{c13}C(O)R^{b13}, NR^{c13}C(O)OR^{a13}, NR^{c13}C(O)NR^{c13}R^{d13}, C(═NR^{e13})R^{b13}, C(═NR^{e13})NR^{c13}R^{d13}, NR^{c13}C(═NR^{e13})NR^{c13}R^{d13}, NR^{c13}C(═NR^{e13})R^{b13}, NR^{c13}S(O)NR^{c13}R^{d13}, NR^{c13}S(O)R^{b13}, NR^{c13}S(O)₂R^{b13}, NR^{c13}S(O)(═NR^{e13})R^{b13}, NR^{c13}S(O)₂NR^{c13}R^{d13}, S(O)R^{b13}, S(O)NR^{c13}R^{d13}, S(O)₂R^{b13}, S(O)₂NR^{c13}R^{d13}, OS(O)(═NR^{e13})R^{b13}, OS(O)₂R^{b13}, S(O)(═NR^{e13})R^{b13}, SF₅, P(O)R^{f13}R^{g1}, OP(O)(OR^{h13})(ORⁱ¹³), P(O)(OR^{h13})(ORⁱ¹³), and BR^{j13}R^{k13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13}, and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c13} and R^{d13} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{G} substituents;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f13} and R^{g13} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h13} and Rⁱ¹³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j13} and R^{k13} is independently selected from OH, C₁₋₆ alkoxy, and C₁₋₆ haloalkoxy;
or any R^{j13} and R^{k13} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from C₁₋₆ alkyl, C₁₋₆ haloalkyl, C₂₋₆ alkenyl, C₂₋₆ alkynyl, Cy², and —(C₁₋₄ alkylene)-Cy², wherein said C₁₋₆ alkyl, C₁₋₆ haloalkyl, C₂₋₆ alkenyl, and C₂₋₆ alkynyl are each optionally substituted with 1, 2, 3, 4, 5, or 6 independently selected R^{2A} substituents;
Cy² is C₃₋₁₀ cycloalkyl, 4-10 membered heterocycloalkyl, 6-10 membered aryl, or 5-10 membered heteroaryl, each of which is optionally substituted with 1, 2, 3, 4, 5, or 6 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂-6 alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{2A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})R^{c21}R^{d21}, NR^{c21}C(═NR^{e21}R^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents;
or two R^{2A} substituents attached to C₃₋₁₀ cycloalkyl, 6-10 membered aryl, 4-10 membered heterocycloalkyl, or 5-10 membered heteroaryl, together with the atoms to which they are connected, form a C₃₋₇ cycloalkyl, phenyl, 4-7 membered heterocycloalkyl, or 5-6 membered heteroaryl ring, which is optionally substituted by 1, 2, 3, or 4 independently selected R^{Cy2} substituents;
each R^{Cy2} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c2}C(O)R^{b21}, NR^{c21}C(O)OR^{a21}, NR^{c21}C(O)NR^{c21}R^{d21}, C(═NR^{e21})R^{b21}, C(═NR^{e21})R^{c21}R^{d21}, NR^{c21}C(═NR^{e21}NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}Rⁱ²¹, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})R^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
or, any R^{c22} and R^{d22} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{2C} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2C} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2C} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a23}, SR^{a23}, NHOR^{a23}, C(O)R^{b23}, C(O)NR^{c23}R^{d23}, C(O)NR^{c23}(OR^{a23}), C(O)OR^{a23}, OC(O)R^{b23}, OC(O)NR^{c23}R^{d23}, NR^{c23}R^{d23}, NR^{c23}NR^{c23}R^{d23}, NR^{c23}C(O)R^{b23}, NR^{c23}C(O)OR^{a23}, NR^{c23}C(O)NR^{c23}R^{d23}, C(═NR^{e23})R^{b23}, C(═NR^{e23})NR^{c23}R^{d23}, NR^{c23}C(═NR^{e23})NR^{c23}R^{d23}, NR^{c23}C(═NR^{e23})R^{b23}, NR^{c23}S(O)NR^{c23}R^{d23}, NR^{c23}S(O)R^{b23}, NR^{c23}S(O)₂R^{b23}, NR^{c23}S(O)(═NR^{e23})R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, OS(O)(═NR^{e23})R^{b23}, OS(O)₂R^{b23}, S(O)(═NR^{e23})R^{b23}, SF₅, P(O)R^{f23}R^{g23}, OP(O)(OR^{h23})(ORⁱ²³), P(O)(OR^{h23})(ORⁱ²³), and BR^{j23}R^{k23}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a23}, R^{c23}, and R^{d23} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c23} and R^{d23} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{G} substituents;
each R^{b23} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f23} and R^{g23} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h23} and Rⁱ²³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j23} and R^{k23} is independently selected from OH, C₁₋₆ alkoxy, and C₁₋₆ haloalkoxy;
or any R^{j23} and R^{k23} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, OR^{a3}, SR^{a3}, NHOR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, S(O)(═NR^{e3})R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3A} substituents;
or, any R^{c3} and R^{d3} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{3A} substituents;
each R^{b3} is independently selected from C₁₋₆ alkyl, C₁₋₆ haloalkyl, C₂₋₆ alkenyl, C₂-6 alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃ C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j3} and R^{k3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a31}, SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}C(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31}, NR^{c31}S(O)NR^{c31}R^{d31}, NR^{c31}S(O)R^{b31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, S(O)(═NR^{e31})R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, and C₃₋₄ cycloalkyl, wherein said C₁₋₆ alkyl, C₁₋₆ haloalkyl, C₂₋₆ alkenyl, C₂₋₆ alkynyl, and C₃₋₄ cycloalkyl are each optionally substituted with 1, 2, 3, or 4 independently selected R^{G} substituents;
or, any R^{c31} and R^{d31} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{G} substituents;
each R^{b31} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f31} and R^{g31} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h31} and Rⁱ³¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j31} and R^{k31} is independently selected from OH, C₁₋₆ alkoxy, and C₁₋₆ haloalkoxy;
or any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁴ is selected from H, halo, cyano, hydroxy, amino, (C₁₋₄ alkyl)amino, di(C₁₋₄ alkyl)amino, C₁₋₄ alkyl, C₁₋₄ haloalkyl, C₁₋₄ alkoxy, and C₁₋₄ haloalkoxy;
R⁵ is selected from H, halo, cyano, hydroxy, amino, (C₁₋₄ alkyl)amino, di(C₁₋₄ alkyl)amino, C₁₋₄ alkyl, C₁₋₄ haloalkyl, C₁₋₄ alkoxy, and C₁₋₄ haloalkoxy;
R⁶ is selected from H, halo, cyano, hydroxy, amino, (C₁₋₄ alkyl)amino, di(C₁₋₄ alkyl)amino, C₁₋₄ alkyl, C₁₋₄ haloalkyl, C₁₋₄ alkoxy, and C₁₋₄ haloalkoxy; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R¹ is —R^{Y}.

In some embodiments, R^{Y} is Cy¹.

In some embodiments, each R^{Cy1} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b1}, NR^{c11}C(O)OR^{a1}, NR^{c11}C(O)NR^{c11}R^{d11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents.

In some embodiments, Cy¹ is 6-10 membered aryl, 5-10 membered heteroaryl, or 6-10 membered heterocycloalkyl each of which is optionally substituted with 1, 2, 3, 4, 5, or 6 independently selected R^{1A} substituents.

In some embodiments, Cy¹ is phenyl, 5-6 membered heteroaryl, or 4-10 membered heterocycloalkyl each of which is optionally substituted with 1, 2, 3, 4, 5, or 6 independently selected R^{1A} substituents.

In some embodiments, Cy¹ is phenyl, 5-6 membered heteroaryl, or 4-10 membered heterocycloalkyl each of which is optionally substituted with 1, 2, 3, or 4 independently selected R^{1A} substituents.

In some embodiments, Cy¹ is phenyl, 5-6 membered heteroaryl, or 4-10 membered heterocycloalkyl each of which is optionally substituted with 1 or 2 independently selected R^{1A} substituents.

In some embodiments, Cy¹ is 6-10 membered aryl, which is optionally substituted with 1, 2, 3, 4, 5, or 6 independently selected R^{1A} substituents.

In some embodiments, Cy¹ is phenyl, which is optionally substituted with 1, 2, 3, or 4 independently selected R^{1A} substituents.

In some embodiments, Cy¹ is phenyl, which is optionally substituted with 1 or 2 independently selected R^{1A} substituents.

In some embodiments, Cy¹ is 5-10 membered heteroaryl, which is optionally substituted with 1, 2, 3, or 4 independently selected R^{1A} substituents.

In some embodiments, Cy¹ is 5-6 membered heteroaryl, which is optionally substituted with 1, 2, 3, or 4 independently selected R^{1A} substituents.

In some embodiments, Cy¹ is 5-6 membered heteroaryl, which is optionally substituted with 1 or 2 independently selected R^{1A} substituents.

In some embodiments, Cy¹ is 4-10 membered heterocycloalkyl, which is optionally substituted with 1, 2, 3, or 4 independently selected R^{1A} substituents.

In some embodiments, Cy¹ is 4-10 membered heterocycloalkyl, which is optionally substituted with 1 or 2 independently selected R^{1A} substituents.

In some embodiments, Cy¹ is phenyl, pyrrolyl, thiophenyl, furanyl, imidazolyl, pyrazolyl, oxazolyl, isoxazolyl, thiazolyl, isothiazolyl, thiadiazolyl, oxadiazolyl, triazolyl, pyridyl, pyrimidinyl, pyridazinyl, pyrazinyl, or oxoisoindolinyl, each of which is optionally substituted with 1, 2, 3, or 4 independently selected R^{1A} substituents.

In some embodiments, Cy¹ is phenyl, pyrazolyl, pyridyl, pyrimidinyl, pyrazinyl, pyridazinyl, or oxoisoindolinyl, each of which is optionally substituted with 1, 2, 3, or 4 independently selected R^{1A} substituents.

In some embodiments, two R^{1A} attached to C₃₋₁₀ cycloalkyl, 6-10 membered aryl, 4-10 membered heterocycloalkyl, or 5-10 membered heteroaryl, together with the atoms to which they are connected substituents together with the atoms to which they are connected, form a 4-7 membered heterocycloalkyl ring, which is optionally substituted by 1, 2, 3, or 4 independently selected R^{Cy1} substituents.

In some embodiments, two R^{1A} attached to 6-10 membered aryl together with the atoms to which they are connected substituents together with the atoms to which they are connected, form a 4-7 membered heterocycloalkyl ring, which is optionally substituted by 1, 2, 3, or 4 independently selected R^{Cy1} substituents.

In some embodiments, two R^{1A} attached to phenyl together with the atoms to which they are connected substituents together with the atoms to which they are connected, form a 4-6 membered heterocycloalkyl ring, which is optionally substituted by 1, 2, or 3 independently selected R^{Cy1} substituents.

In some embodiments, each R^{1A} is independently selected from halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11} NR^{c11}C(O)OR^{a11}, NR^{c11}C(O)NR^{c11}R^{d11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents.

In some embodiments, each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents.

In some embodiments, each R^{1A} is independently selected from halo, CN, NO₂, C₁₋₆ alkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b11}, C(O)NR^{c11}R^{d11}, and NR^{c11}C(O)R^{b11}, wherein said C₁₋₆ alkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents.

In some embodiments, each R^{1A} is independently selected from halo, C₃₋₁₀ cycloalkyl, 4-10 membered heterocycloalkyl, 4-10 membered heterocycloalkyl-C₁₋₄ alkyl, C(O)R^{b11}, C(O)NR^{c11}R^{d11}, and NR^{c11}C(O)R^{b11}, wherein said C₃₋₁₀ cycloalkyl, 4-10 membered heterocycloalkyl, and 4-10 membered heterocycloalkyl-C₁₋₄ alkyl are each optionally substituted with 1, 2, 3, or 4 independently selected R^{1B} substituents.

In some embodiments, each R^{1A} is independently selected from halo, C₃₋₇ cycloalkyl, 4-7 membered heterocycloalkyl, 4-7 membered heterocycloalkyl-C₁₋₄ alkyl, C(O)R^{b11}, C(O)NR^{c11}R^{d11}, and NR^{c11}C(O)R^{b11}, wherein said C₃₋₇ cycloalkyl, 4-7 membered heterocycloalkyl, and 4-7 membered heterocycloalkyl-C₁₋₄ alkyl are each optionally substituted with 1, 2, 3, or 4 independently selected R^{1B} substituents.

In some embodiments, each R^{1A} is independently selected from halo, C₃₋₇ cycloalkyl, 4-7 membered heterocycloalkyl, 4-7 membered heterocycloalkyl-C₁₋₄ alkyl, C(O)R^{b11}, C(O)NR^{c11}R^{d11}, and NR^{c11}C(O)R^{b11}, wherein said C₃₋₇ cycloalkyl, 4-7 membered heterocycloalkyl, and 4-7 membered heterocycloalkyl-C₁₋₄ alkyl are each optionally substituted with 1 or 2 independently selected R^{1B} substituents.

In some embodiments, each R^{1A} is independently selected from fluoro, CN, isopropyl, cyclopropyl, cyclobutyl, piperazinyl, tetrahydropyranyl, CH₂-pyrrolidinyl, C(O)N(CH₃)₂, NHC(O)CH₃, C(O)-morpholinyl, C(O)-azetidinyl, C(O)-piperazinyl, C(O)-pyrrolidinyl, and C(O)-(7-10 membered heterocycloalkyl), wherein the isopropyl, cyclopropyl, cyclobutyl, piperazinyl, tetrahydropyranyl, CH₂-pyrrolidinyl, C(O)-morpholinyl, C(O)-azetidinyl, C(O)-piperazinyl, C(O)-pyrrolidinyl, and C(O)-(7-10 membered heterocycloalkyl) are each optionally substituted with 1 or 2 substituents independently selected from methyl, OH, OCH₃, and amino.

In some embodiments, each R^{c11} and R^{d11} is independently selected from H and C₁₋₆ alkyl, or, any R^{c11} and R^{d11} attached to the same N atom, together with the N atom to which they are attached, form a 4-10 membered heterocycloalkyl group, wherein the 6-membered heteroaryl or 4-10 membered heterocycloalkyl group is optionally substituted with 1 or 2 independently selected R^{1B} substituents.

In some embodiments:
each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-10 membered heterocycloalkyl group, which is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)OR^{a12}, OC(O)R^{b12}, OC(O)NR^{b12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12} and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents;
each R^{1C} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, C(O)R^{b13}, C(O)NR^{c13}R^{d13}, C(O)OR^{a1}, OC(O)R^{b13}, OC(O)NR^{c13}R^{d13}, NR^{c13}R^{d13}, NR^{c13}C(O)R^{b13}, NR^{c13}C(O)OR^{a13}, NR^{c13}C(O)NR^{c13}R^{d13}, NR^{c13}S(O)₂R^{b13}, NR^{c13}S(O)₂NR^{c13}R^{d13}, S(O)₂R^{b13}, and S(O)₂NR^{c13}R^{d13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13}, and R^{d13} is independently selected from H, C₁₋₆ alkyl, and C₁₋₆ haloalkyl; and
each R^{b13} is independently selected from C₁₋₆ alkyl and C₁₋₆ haloalkyl.

In some embodiments:
each R^{a11}, R^{c11}, and R^{d11} is independently selected from H, C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-10 membered heterocycloalkyl group, which is optionally substituted with 1, 2, 3, or 4 independently selected R^{1B} substituents;
each R^{b11} is independently selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{1B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected Ric substituents;
each R^{a12}, R^{c12} and R^{d12} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{b12} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents;
each R^{1C} is independently selected from H, D, halo, CN, NO₂, C₁₋₆ alkyl, C₁₋₆ haloalkyl, C₃₋₄ cycloalkyl, OR^{a13}, SR^{a13}, C(O)R^{b13}, C(O)NR^{c13}R^{d13}, C(O)OR^{a13}, OC(O)R^{b13}, OC(O)NR^{c13}R^{d13}, NR^{c13}R^{d13}, NR^{c13}C(O)R^{b13}, NR^{c13}C(O)OR^{a13}, NR^{c13}C(O)NR^{c13}R^{d13}, NR^{c13}S(O)₂R^{b13}, NR^{c13}S(O)₂NR^{c13}R^{d13}, S(O)₂R^{b13}, and S(O)₂NR^{c13}R^{d13};
each R^{a13}, R^{c13}, and R^{d13} is independently selected from H, C₁₋₆ alkyl, and C₁₋₆ haloalkyl; and
each R^{b13} is independently selected from C₁₋₆ alkyl and C₁₋₆ haloalkyl.

In some embodiments:
each R^{a11}, R^{c11}, and R^{d11} is independently selected from H, C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-10 membered heterocycloalkyl group, which is optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12} and R^{d12} is independently selected from H, C₁₋₆ alkyl, and C₁₋₆ haloalkyl; and
each R^{b12} is independently selected from C₁₋₆ alkyl and C₁₋₆ haloalkyl.

In some embodiments:
each R^{a11}, R^{c11}, and R^{d11} is independently selected from H, C₁₋₆ alkyl, and 4-7 membered heterocycloalkyl, wherein said C₁₋₆ alkyl and 4-7 membered heterocycloalkyl are each optionally substituted with 1, 2, or 3 independently selected R^{1B} substituents;
or, any R^{c11} and R^{d11} attached to the same N atom, together with the N atom to which they are attached, form a 4-10 membered heterocycloalkyl group, which is optionally substituted with 1, 2, or 3 independently selected R^{1B} substituents;
each R^{b11} is independently C₁₋₆ alkyl, which is optionally substituted with 1, 2, 3, or 4 independently selected R^{1B} substituents;
each R^{1B} is independently C₁₋₆ alkyl, OR^{a12}, and NR^{c12}R^{d12}; and
each R^{a12}, R^{c12} and R^{d12} is independently selected from H and C₁₋₆ alkyl.

In some embodiments, R^{1B} is independently selected from C₁₋₆ alkyl, OR^{a12}, and NR^{c12}R^{d12}, wherein each R^{a12}, R^{c12}, and R^{d12} is independently selected from H, C₁₋₆ alkyl, and C₁₋₆ haloalkyl.

In some embodiments, R^{1B} is independently selected from methyl, OH, OCH₃, and amino.

In some embodiments, R² is Cy².

In some embodiments, Cy² is C₃₋₇ cycloalkyl, 4-7 membered heterocycloalkyl, phenyl, or 5-6 membered heteroaryl, each of which is optionally substituted with 1, 2, 3, or 4 independently selected R^{2A} substituents.

In some embodiments, Cy² is C₃₋₁₀ cycloalkyl or 4-10 membered heterocycloalkyl, each of which is optionally substituted with 1, 2, 3, or 4 independently selected R^{2A} substituents.

In some embodiments, Cy² is C₃₋₇ cycloalkyl or 4-7 membered heterocycloalkyl, each of which is optionally substituted with 1, 2, 3, or 4 independently selected R^{2A} substituents.

In some embodiments, Cy² is C₃₋₁₀ cycloalkyl, which is optionally substituted with 1, 2, 3, or 4 independently selected R^{2A} substituents.

In some embodiments, Cy² is C₃₋₇ cycloalkyl, which is optionally substituted with 1, 2, 3, or 4 independently selected R^{2A} substituents.

In some embodiments, Cy² is C₃₋₇ cycloalkyl, which is optionally substituted with 1 or 2 independently selected R^{2A} substituents.

In some embodiments, Cy² is C₃₋₇ cycloalkyl, which is optionally substituted with 1 R^{2A} substituent.

In some embodiments, Cy² is cyclopropyl, cyclobutyl, cyclopentyl, or cyclohexyl, each of which is optionally substituted with 1, 2, 3, or 4 independently selected R^{2A} substituents.

In some embodiments, Cy² is cyclopropyl, cyclobutyl, cyclopentyl, or cyclohexyl, each of which is optionally substituted with 1 or 2 independently selected R^{2A} substituents.

In some embodiments, Cy² is cyclopropyl, cyclobutyl, cyclopentyl, or cyclohexyl, each of which is optionally substituted with 1 R^{2A} substituent.

In some embodiments, Cy² is cyclohexyl, which is optionally substituted with 1 or 2 independently selected R^{2A} substituents.

In some embodiments, Cy² is cyclohexyl, which is optionally substituted with 1 R^{2A} substituent.

In some embodiments, Cy² is cyclohexyl, which is substituted with —CH₂CN.

In some embodiments, each R^{2A} is independently selected from halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21} NR^{c21}C(O)OR^{a21}, NR^{c21}C(O)NR^{c21}R^{d21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents.

In some embodiments, each R^{2A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents.

In some embodiments, each R^{2A} is independently selected from C₁₋₆ alkyl, C₂₋₆ alkenyl, C₂₋₆ alkynyl, and C₁₋₆ haloalkyl, wherein said C₁₋₆ alkyl, C₂₋₆ alkenyl, C₂₋₆ alkynyl, and C₁₋₆ haloalkyl are each optionally substituted with 1 or 2 independently selected R^{2B} substituents.

In some embodiments, each R^{2A} is C₁₋₆ alkyl, which is optionally substituted with 1 or 2 independently selected R^{2B} substituents.

In some embodiments, each R^{2A} is C₁₋₆ alkyl, which is optionally substituted with 1 R^{2B} substituent.

In some embodiments, each R^{2A} is methylene, which is optionally substituted with 1 R^{2B} substituent.

In some embodiments, R^{2B} substituent is CN.

In some embodiments:
each R^{a21}, R^{c21}, and R^{d21} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{2B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c22}R^{d22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2C} substituents;
each R^{2C} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a23}, SR^{a23}, C(O)R^{b23}, C(O)NR^{c23}R^{d23}, C(O)OR^{a23}, OC(O)R^{a23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)₂R^{b23}, NR^{c23}S(O)₂NR^{c23}R^{d23}, S(O)₂R^{b23}, and S(O)₂NR^{c23}R^{d23}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a23}, R^{c23}, and R^{d23} is independently selected from H, C₁₋₆ alkyl, and C₁₋₆ haloalkyl; and
each R^{b23} is independently selected from C₁₋₆ alkyl and C₁₋₆ haloalkyl.

In some embodiments:
each R^{a21}, R^{c21}, and R^{d21} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{b21} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{2B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄alkyl, 5-6 membered heteroaryl-C₁₋₄ alkyl, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c22}R^{d22}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
each R^{a22}, R^{c22}, and R^{d22} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
each R^{b22} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2C} substituents;
each R^{1C} is independently selected from H, D, halo, CN, NO₂, C₁₋₆ alkyl, C₁₋₆ haloalkyl, C₃₋₄ cycloalkyl, OR^{a23}, SR^{a23}, C(O)R^{b23}, C(O)NR^{c23}R^{d23}, C(O)OR^{a23}, OC(O)R^{b23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)₂R^{b23}, NR^{c23}S(O)₂NR^{c23}R^{d23}, S(O)₂R^{b23}, and S(O)₂NR^{c23}R^{d23};
each R^{a23}, R^{c23}, and R^{d23} is independently selected from H, C₁₋₆ alkyl, and C₁₋₆ haloalkyl; and
each R^{b23} is independently selected from C₁₋₆ alkyl and C₁₋₆ haloalkyl.

In some embodiments:
each R^{a21}, R^{c21}, and R^{d21} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{b21} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{2B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c21}R^{d21};
each R^{a22}, R^{c22}, and R^{d22} is independently selected from H, C₁₋₆ alkyl, and C₁₋₆ haloalkyl; and
each R^{b22} is independently selected from C₁₋₆ alkyl and C₁₋₆ haloalkyl.

In some embodiments:
each R^{a21}, R^{c21}, and R^{d21} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{b21} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{2B} is independently selected from H, D, halo, CN, NO₂, C₁₋₆ alkyl, C₁₋₆ haloalkyl, C₃₋₄ cycloalkyl, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c22}R^{d22};
each R^{a22}, R^{c22}, and R^{d22} is independently selected from H, C₁₋₆ alkyl, and C₁₋₆ haloalkyl; and
each R^{b22} is independently selected from C₁₋₆ alkyl and C₁₋₆ haloalkyl.

In some embodiments:
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from H, D, halo, CN, NO₂, C₁₋₆ alkyl, C₁₋₆ haloalkyl, C₃₋₄ cycloalkyl, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c22}R^{d22};
each R^{a22}, R^{c22}, and R^{d22} is independently selected from H, C₁₋₆ alkyl, and C₁₋₆ haloalkyl; and
each R^{b22} is independently selected from C₁₋₆ alkyl and C₁₋₆ haloalkyl.

In some embodiments:
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from halo, CN, OR^{a22}, and NR^{c22}R^{d22}; and
each R^{a22}, R^{c22}, and R^{d22} is independently selected from H and C₁₋₆ alkyl.

In some embodiments, R³ is selected from H, D, halo, CN, C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl, OR^{a3}, SR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl are each optionally substituted by 1, 2, 3, or 4 independently selected R^{3A} substituents.

In some embodiments, R³ is selected from C₁₋₆ alkyl, C₁₋₆ haloalkyl, C₂₋₆ alkenyl, C₂₋₆ alkynyl, and C₃₋₆ cycloalkyl, wherein said C₁₋₆ alkyl, C₁₋₆ haloalkyl, C₂₋₆ alkenyl, C₂₋₆ alkynyl, and C₃₋₆ cycloalkyl are each optionally substituted by 1, 2, 3, or 4 independently selected R^{3A} substituents.

In some embodiments, R³ is selected from C₁₋₆ alkyl and C₁₋₆ haloalkyl, which are each optionally substituted by 1 or 2 independently selected R^{3A} substituents.

In some embodiments, R³ is selected from C₁₋₆ alkyl and C₁₋₆ haloalkyl, which are each optionally substituted by 1 R^{3A} substituent.

In some embodiments, each R^{3A} is independently selected from H, D, halo, CN, NO₂, OR^{a31}, C₁₋₆ alkyl, C₂₋₆ alkenyl, C₂₋₆ alkynyl, and C₁₋₆ haloalkyl.

In some embodiments, each R^{3A} is H or OR^{a31}.

In some embodiments, each R^{a31} is independently selected from H, C₁₋₆ alkyl, C₁₋₆ haloalkyl, C₂₋₆ alkenyl, and C₂₋₆ alkynyl.

In some embodiments, each R^{a31} is independently selected from H and C₁₋₆ alkyl.

In some embodiments, each R^{a31} is independently selected from H and C₁₋₃ alkyl.

In some embodiments, R³ is selected from C₁₋₆ alkyl and C₁₋₆ haloalkyl, wherein the C₁₋₆ alkyl and C₁₋₆ haloalkyl are each optionally substituted by 1 or 2 independently selected OR^{a31} substituents, wherein each R^{a31} is independently selected from H and C₁₋₆ alkyl.

In some embodiments:
each R^{a3}, R^{c3}, and R^{d3} is independently selected from H, C₁₋₆ alkyl, and C₁₋₆ haloalkyl, wherein the C₁₋₆ alkyl and C₁₋₆ haloalkyl are each optionally substituted with 1, 2, 3, or 4 independently selected R^{3A} substituents;
each R^{b3} is independently selected from C₁₋₆ alkyl and C₁₋₆ haloalkyl, which are each optionally substituted with 1, 2, 3, or 4 independently selected R^{3A} substituents;
each R^{3A} is independently selected from H, D, halo, CN, C₁₋₆ alkyl, C₁₋₆ haloalkyl, OR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{c31}C(O)OR^{a31}, NR^{c31}C(O)NR^{c31}R^{d31}, NR^{c31}S(O)₂R^{b31}, NR^{c31}S(O)₂NR^{c31}R^{d31}, S(O)₂R^{b31}, and S(O)₂NR^{c31}R^{d31}, wherein said C₁₋₆ alkyl and C₁₋₆ haloalkyl are each optionally substituted with 1, 2, 3, or 4 independently selected R^{G} substituents;
each R^{a31}, R^{c31}, and R^{d31} is independently selected from H, C₁₋₆ alkyl, and C₁₋₆ haloalkyl, wherein the C₁₋₆ alkyl and C₁₋₆ haloalkyl are each optionally substituted with 1, 2, 3, or 4 independently selected R^{G} substituents; and
each R^{b31} is independently selected from C₁₋₆ alkyl and C₁₋₆ haloalkyl, which are each optionally substituted with 1, 2, 3, or 4 independently selected R^{G} substituents.

In some embodiments:
each R^{a3}, R^{c3}, and R^{d3} is independently selected from H, C₁₋₆ alkyl, and C₁₋₆ haloalkyl, wherein the C₁₋₆ alkyl and C₁₋₆ haloalkyl are each optionally substituted with 1, 2, 3, or 4 independently selected R^{3A} substituents;
each R^{b3} is independently selected from C₁₋₆ alkyl and C₁₋₆ haloalkyl, which are each optionally substituted with 1, 2, 3, or 4 independently selected R^{3A} substituents;
each R^{3A} is independently selected from H, D, halo, CN, C₁₋₆ alkyl, C₁₋₆ haloalkyl, OR^{a31}, and NR^{c31}R^{d31};
each R^{a31}, R^{c31}, and R^{d31} is independently selected from H, C₁₋₆ alkyl, and C₁₋₆ haloalkyl;
each R^{b31} is independently selected from C₁₋₆ alkyl and C₁₋₆ haloalkyl.

In some embodiments, each R^{3A} is independently selected from H, D, halo, CN, C₁₋₆ alkyl, C₁₋₆ haloalkyl, OR^{a31}, and NR^{c31}R^{d31}, wherein each R^{a31}, R^{c31}, and R^{d31} is independently selected from H, C₁₋₆ alkyl, and C₁₋₆ haloalkyl.

In some embodiments, each R^{3A} is independently selected from halo, CN, OR^{a31}, and NR^{c31}R^{d31}, wherein each R^{a31}, R^{c31}, and R^{d31} is independently selected from H, C₁₋₆ alkyl, and C₁₋₆ haloalkyl.

In some embodiments, each R^{3A} is independently selected from halo, CN, OR^{a31}, and NR^{c31}R^{d31}, wherein each R^{a31}, R^{c31}, and R^{d31} is independently selected from H or methyl.

In some embodiments, each R^{3A} is OH.

In some embodiments, R³ is selected from C₁₋₄ alkyl and C₁₋₄ haloalkyl wherein the C₁₋₄ alkyl and C₁₋₄ haloalkyl are each optionally substituted by 1 or 2 independently selected OR^{a31} substituents, wherein each R^{a31} is independently selected from H and C₁₋₆ alkyl.

In some embodiments, R³ is C₁₋₆ alkyl, wherein said C₁₋₆ alkyl is optionally substituted by 1, 2, 3, or 4 independently selected R^{3A} substituents.

In some embodiments, R³ is C₁₋₄ alkyl, wherein said C₁₋₄ alkyl is optionally substituted by 1 or 2 independently selected R^{3A} substituents.

In some embodiments, R³ is C₁₋₃ alkyl, wherein said C₁₋₃ alkyl is optionally substituted by 1 or 2 independently selected R^{3A} substituents.

In some embodiments, R³ is C₁₋₂ alkyl, which is optionally substituted by OR^{a1} wherein the R^{a31} is selected from H and C₁₋₃ alkyl.

In some embodiments, R³ is C₁₋₆ alkyl, which is optionally substituted by OH.

In some embodiments, R³ is C₁₋₄ alkyl, which is optionally substituted by OH.

In some embodiments, R³ is C₁₋₃ alkyl, which is optionally substituted by OH.

In some embodiments, R³ is C₁₋₂ alkyl, which is optionally substituted by OH.

In some embodiments, R³ is methyl or 2-hydroxyethyl.

In some embodiments, R³ is methyl.

In some embodiments, R³ is 2-hydroxyethyl.

In some embodiments, R⁴ is selected from H, halo, CN, hydroxy, amino, C₁₋₃ alkyl, and C₁₋₃ haloalkyl.

In some embodiments, R⁴ is selected from H, halo, and CN.

In some embodiments, R⁴ is H.

In some embodiments, R⁵ is selected from H, halo, CN, hydroxy, amino, C₁₋₃ alkyl, and C₁₋₃ haloalkyl.

In some embodiments, R⁵ is selected from H, halo, and CN.

In some embodiments, R⁵ is H.

In some embodiments, R⁶ is selected from H, halo, CN, hydroxy, amino, C₁₋₃ alkyl, and C₁₋₃ haloalkyl.

In some embodiments, R⁶ is selected from H, halo, and CN.

In some embodiments, R⁶ is H.

In some embodiments, R⁴, R⁵, and R⁶ are each H.

In some embodiments:
R³ is C₁₋₃ alkyl, which is optionally substituted with OH; and
R⁴, R⁵, and R⁶ are each H.

In some embodiments:
R¹ is —R^{Y};
R is Cy¹;
Cy¹ is phenyl, 5-6 membered heteroaryl, or 4-10 membered heterocycloalkyl each of which is optionally substituted with 1, 2, 3, or 4 independently selected R^{1A} substituents;
each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, N^{c11}C(O)NR^{c11}R^{d11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-10 membered heterocycloalkyl group, which is optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12} and R^{d12} is independently selected from H, C₁₋₆ alkyl, and C₁₋₆ haloalkyl;
each R^{b12} is independently selected from C₁₋₆ alkyl and C₁₋₆ haloalkyl;
R² is Cy²;
Cy² is C₃₋₁₀ cycloalkyl or 4-10 membered heterocycloalkyl, each of which is optionally substituted with 1, 2, 3, or 4 independently selected R^{2A} substituents;
each R^{2A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R³ is selected from H, D, halo, CN, C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl, OR^{a3}, SR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl are each optionally substituted by 1, 2, 3, or 4 independently selected R^{3A} substituents;
each R^{3A} is independently selected from halo, CN, OR^{a31}, and NR^{c31}R^{d31}, wherein each R^{a31}, R^{c31}, and R^{d31} is independently selected from H, C₁₋₆ alkyl, and C₁₋₆ haloalkyl;
each R^{a31}, R^{c31}, and R^{d31} is independently selected from H, C₁₋₆ alkyl, and C₁₋₆ haloalkyl;
R⁴ is selected from H, halo, CN, hydroxy, amino, C₁₋₃ alkyl, and C₁₋₃ haloalkyl;
R⁵ is selected from H, halo, CN, hydroxy, amino, C₁₋₃ alkyl, and C₁₋₃ haloalkyl; and R⁶ is selected from H, halo, CN, hydroxy, amino, C₁₋₃ alkyl, and C₁₋₃ haloalkyl;

In some embodiments:
R¹ is —R^{Y};
R is Cy¹;
Cy¹ is phenyl, 5-6 membered heteroaryl, or 4-10 membered heterocycloalkyl each of which is optionally substituted with 1, 2, 3, or 4 independently selected R^{1A} substituents;
each R^{a11}, R^{c11}, and R^{d11} is independently selected from H, C₁₋₆ alkyl, and 4-7 membered heterocycloalkyl, wherein said C₁₋₆ alkyl and 4-7 membered heterocycloalkyl are each optionally substituted with 1, 2, or 3 independently selected R^{1B} substituents;
or, any R^{c11} and R^{d11} attached to the same N atom, together with the N atom to which they are attached, form a 4-10 membered heterocycloalkyl group, which is optionally substituted with 1, 2, or 3 independently selected R^{1B} substituents;
each R^{b11} is independently C₁₋₆ alkyl, which is optionally substituted with 1, 2, 3, or 4 independently selected R^{1B} substituents;
each R^{1B} is independently C₁₋₆ alkyl, OR^{a12}, and NR^{c12}R^{d12};
each R^{a12}, R^{c21} and R^{d12} is independently selected from H and C₁₋₆ alkyl;
R² is Cy²;
Cy² is C₃₋₇ cycloalkyl, which is optionally substituted with 1 or 2 independently selected R^{2A} substituents;
each R^{2A} is C₁₋₆ alkyl, which is optionally substituted with 1 or 2 independently selected R^{2B} substituents;
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R³ is selected from C₁₋₆ alkyl and C₁₋₆ haloalkyl, which are each optionally substituted by 1 or 2 independently selected R^{3A} substituents;
each R^{3A} is independently selected from halo, CN, OR^{a31}, and NR^{c31}R^{d31};
each R^{a31}, R^{c31}, and R^{d31} is independently selected from H, C₁₋₆ alkyl, and C₁₋₆ haloalkyl;
R⁴ is selected from H, halo, and CN;
R⁵ is selected from H, halo, and CN; and
R⁶ is selected from H, halo, and CN;

In some embodiments, the compound or the salt is a compound of Formula II:

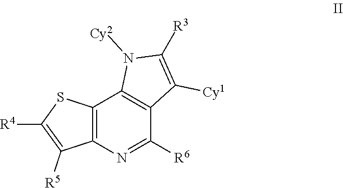

or a pharmaceutically acceptable salt thereof, wherein R³, R⁴, R⁵, R⁶, Cy¹ and Cy² are as defined herein.

In some embodiments, the compound or the salt is a compound of Formula IIa:

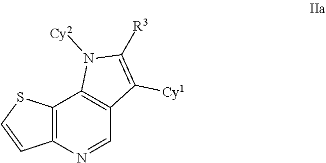

or a pharmaceutically acceptable salt thereof, wherein R³, Cy¹ and Cy² are as defined herein.

In some embodiments, the compound or the salt is a compound of Formula III:

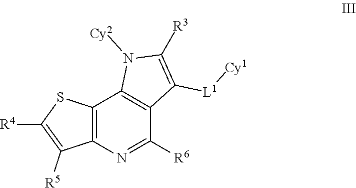

or a pharmaceutically acceptable salt thereof, wherein R³, R⁴, R⁵, R⁶, L¹, Cy¹ and Cy² are as defined herein.

In some embodiments, the compound or the salt is a compound of Formula IIIa:

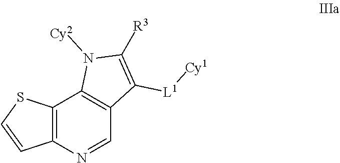

or a pharmaceutically acceptable salt thereof, wherein R³, L¹, Cy¹ and Cy² are as defined herein.

In some embodiments, the compound or the salt is a compound of Formula IV:

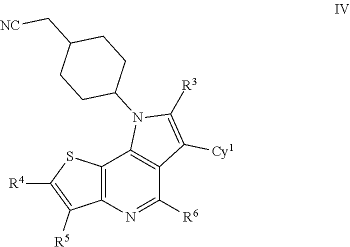

or a pharmaceutically acceptable salt thereof, wherein R³, R⁴, R⁵, R⁶, and Cy¹ are as defined herein.

In some embodiments, the compound or the salt is a compound of Formula IVa:

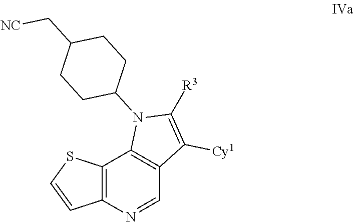

or a pharmaceutically acceptable salt thereof, wherein R³ and Cy¹ are as defined herein.

In some embodiments, the compound or the salt is a compound of Formula V:

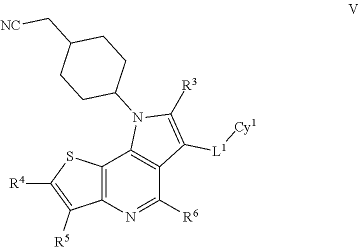

or a pharmaceutically acceptable salt thereof, wherein R³, R⁴, R⁵, R⁶, L¹, and Cy¹ are as defined herein.

In some embodiments, the compound or the salt is a compound of Formula V:

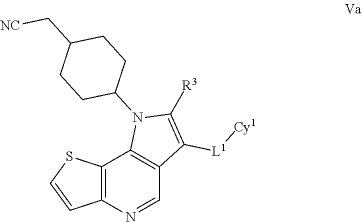

or a pharmaceutically acceptable salt thereof, wherein R³, L¹, and Cy¹ are as defined herein.

In some embodiments, the compound or the salt is a compound of the Examples (infra), or a pharmaceutically acceptable salt thereof. In some embodiments, the compound or the salt is a compound of the Examples (infra). In some embodiments, the compound or the salt is a pharmaceutically acceptable salt of one of the compounds of the Examples (infra).

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ-includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “thio” refers to a group of formula —SH.

As used herein, the term “carbamyl” to a group of formula —C(O)NH₂.

As used herein, the term “carbonyl”, employed alone or in combination with other terms, refers to a —C(O)— group.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “cyano-C₁₋ₙ alkyl” refers to a group of formula —(C₁₋ₙ alkylene)-CN, wherein the alkyl group has 1 to n carbon atoms. In some embodiments, the alkyl group has 1 to 6, 1 to 4, or 1 to 3 carbon atoms, e.g., —(C₁₋₃ alkylene)-CN.

As used herein, the term “HO—C₁₋ₙ alkyl” refers to a group of formula —(C₁₋ₙ alkylene)-OH, wherein the alkyl group has 1 to n carbon atoms. In some embodiments, the alkyl group has 1 to 6, 1 to 4, or 1 to 3 carbon atoms, e.g., —(C₁₋₃ alkylene)-OH.

As used herein, the term “C₁₋ₙ alkoxy-C₁₋ₙ alkyl” refers to a group of formula —(C₁₋ₙ alkylene)-O(C₁₋ₙ alkyl), wherein the alkyl group has 1 to n carbon atoms. In some embodiments, the alkyl group has 1 to 6, 1 to 4, or 1 to 3 carbon atoms, e.g., —(C₁₋₆ alkylene)-O(C₁₋₆ alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkylcarbonyloxy” is a group of formula —OC(O)— alkyl, wherein the alkyl group has n to m carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH— alkyl, wherein the alkyl group has n to m carbon atoms.

As used herein, “di(Cₙ₋ₘalkyl)aminocarbonyloxy” is a group of formula —OC(O)—N(alkyl)₂, wherein each alkyl group has, independently, n to m carbon atoms.

As used herein “Cₙ₋ₘ alkoxycarbonylamino” refers to a group of formula —NHC(O)O(Cₙ₋ₘ alkyl), wherein the alkyl group has n to m carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1, 2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C=O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{1A}) are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

The compounds provided herein, including salts and stereoisomers thereof, can be prepared using known organic synthesis techniques and can be synthesized according to any of numerous possible synthetic routes.

For example, pyrrolo[2,3-d]thieno[3,2-b]pyridine compounds of Formula I can be prepared according to the procedure described in Scheme 1. Thieno[3,2-b]pyridin-7-amine (2) can be obtained via palladium-catalyzed amination of an appropriately substituted chlorothieno[3,2-b]pyridine (1). Thieno[3,2-b]pyridin-7-amine (2) can be converted to the corresponding 6-iodothieno[3,2-b]pyridin-7-amine (3) by treatment with NIS. Palladium-catalyzed annulation of compound 3 with allyl acetate affords compound 4 which can be converted to compound 5 upon treatment with NBS. The substitution at R¹ (6) can be introduced using methods known to one skilled in the art (such as Suzuki cross-coupling, Buchwald-Hartwig cross coupling, etc.).

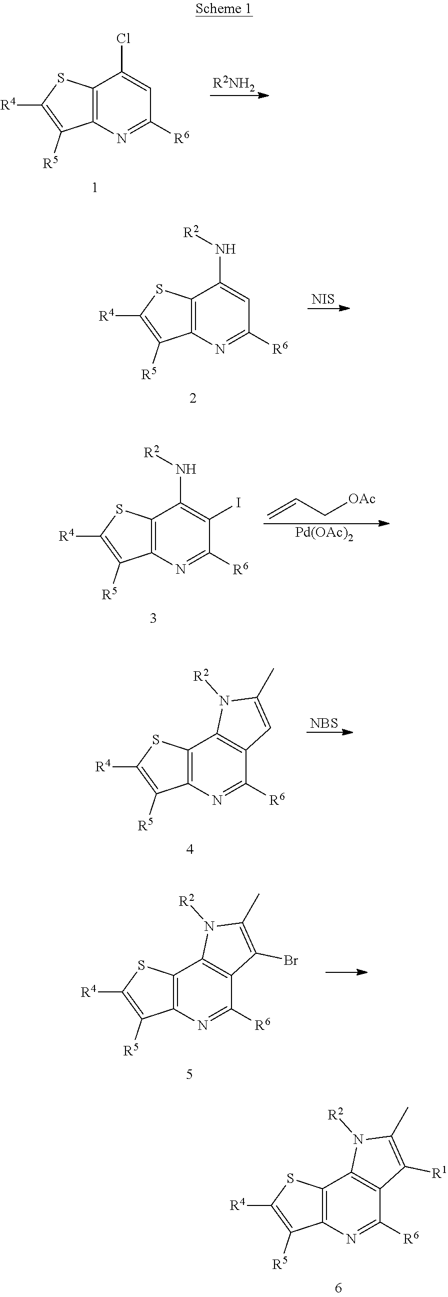

Pyrrolo[2,3-d]thieno[3,2-b]pyridine derivatives of Formula I can also be prepared, for example, according to the procedures described in Scheme 2. Thieno[3,2-b]pyridin-7-amine (2) can be obtained via palladium-catalyzed amination of an appropriately substituted chlorothieno[3,2-b]pyridine (1). Thieno[3,2-b]pyridin-7-amine (2) can be converted to the corresponding 6-iodothieno[3,2-b]pyridin-7-amine (3) by treatment with NIS. Sonogashira cross-coupling of compound 3 with an appropriate alkyne affords compound 7 which can be cyclized to give compound 8 upon treatment with TBAF or CuI. Subsequently, compound 8 can be converted to compound 9 by treatment with NBS. The substitution at R¹ (10) can be introduced using methods known to one skilled in the art (such as Suzuki cross-coupling, Buchwald-Hartwig cross coupling, etc.).

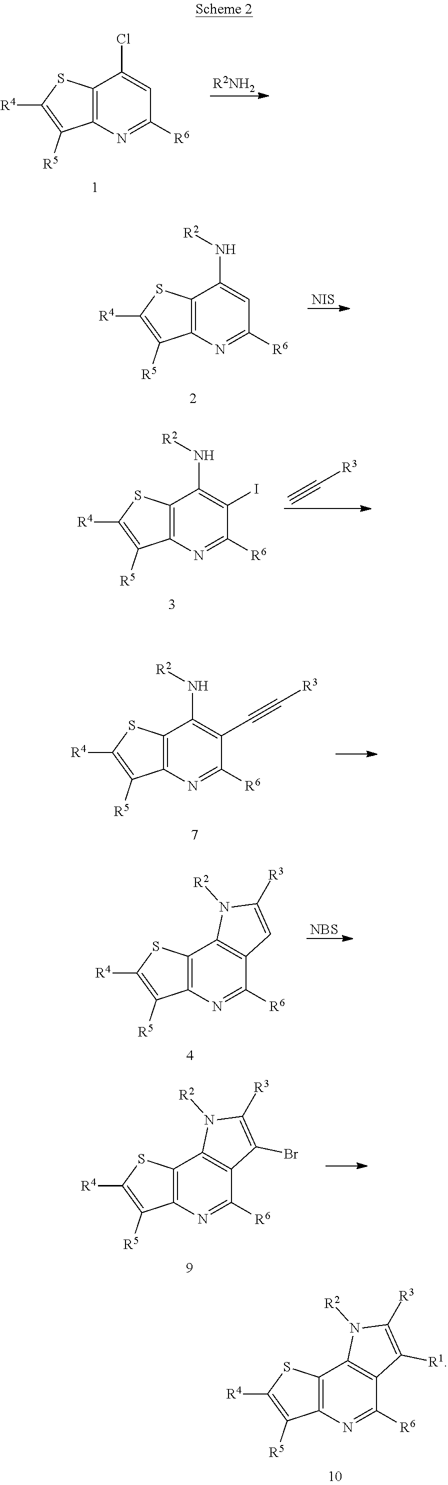

The reactions for preparing compounds provided herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature”, “room temperature”, and “r.t.”, as used herein, are understood in the art, and refer generally to a temperature, e.g. a reaction temperature, that is about the temperature of the room in which the reaction is carried out, for example, a temperature from about 20° C. to about 30° C.

Preparation of compounds provided herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein are inhibitors of TYK2 and/or JAK1 kinases.

In some embodiments, the present application provides a method for treating a disease or disorder associated with JAK1 and/or TYK2 in a patient in need thereof, comprising the step of administering to said patient a compound provided herein, or a pharmaceutically acceptable salt thereof. In some embodiments, the method comprises administering to the patient a pharmaceutical composition comprising a compound provided herein (e.g., a compound of Formula I, or a pharmaceutically acceptable salt), and a pharmaceutically acceptable carrier.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

In some embodiments, the administration is topical administration.

In some embodiments, the administration is oral administration.

In some embodiments, the disease or disorder is selected from a skin disease or disorder, an ophthalmic disease or disorder, a respiratory disease or disorder, and a rectal disease or disorder.

In some embodiments, the disease or disorder is a skin disease or disorder.

In some embodiments, the skin disease or disorder is selected from eczema, atopic dermatitis, psoriasis, contact dermatitis, vitiligo (including non-segmental vitiligo), hidradenitis suppurativa, prurigo nodularis, ichthyosis, lichen planus, lichen sclerosus, alopecia, rosacea, urticaria, non-melanoma skin cancer, dermatomyositis, bullous pemphigoid, pemphigus vulgaris, cutaneous graft-versus-host disease, cutaneous manifestations of interferonopathies, STAT1 gain-of-function, STAT3 gain-of-function, systemic lupus erythematosus, including cutaneous lupus, and scleroderma.

In some embodiments, the disease or disorder is an ophthalmic disease or disorder.

In some embodiments, the ophthalmic disease or disorder is selected from ocular graft-versus-host disease, dry eye, uveitis, scleritis, Behcet's disease, conjunctivitis, and Sjögren's syndrome.

In some embodiments, the disease or disorder is a respiratory disease or disorder.

In some embodiments, the respiratory disease or disorder is selected from asthma, chronic obstructive pulmonary disease, rhinitis, and idiopathic pulmonary fibrosis.

In some embodiments, the disease or disorder is selected from a rectal disease or disorder.

In some embodiments, the rectal disease or disorder is selected from ulcerative colitis, Crohn's disease, celiac disease, Peutz-Jeghers syndrome, juvenile polyposis syndrome, and Cowden Syndrome.

In some embodiments, the disease or disorder is neuroinflammation. In some embodiments, the neuroinflammation is amyotrophic lateral sclerosis (ALS), frontotemporal dementia (FTD), Alzheimer's disease, multiple sclerosis or Parkinson's disease. For example, Alzheimer's disease is the most common form of neurodegenerative disease, estimated to contribute to 60-70% of all cases of dementia worldwide. According to the prevailing amyloid cascade hypothesis, amyloid-β (Aβ) deposition in the brain is the initiating event in Alzheimer's disease, although evidence is accumulating that this hypothesis is insufficient to explain many aspects of Alzheimer's disease pathogenesis. The discovery of increased levels of inflammatory markers in patients with Alzheimer's disease and the identification of Alzheimer's disease risk genes associated with innate immune functions suggest that neuroinflammation has a prominent role in the pathogenesis of Alzheimer's disease (Leng and Edison, Nat Rev Neurol. 2021 March; 17(3):157-172). In particular, the JAK-STAT pathway has been implicated as a target for Alzheimer's disease based on genetic and real-world clinical data (Nevado-Holgado et al. Cells 2019, 8(5)). Type I interferon signaling, regulated by TYK2 and JAK1, has been shown to promote memory impairment associated with amyloid-β plaques (Roy et al. Immunity, 2022, 55, 879) or induce neuronal cell death (Rodriguez, S. et al. Nat Commun. 2021 Feb. 15; 12(1):1033; Rodriguez, S. et al. Sci Transl Med. 2021, 13(610):eaaz4699). Further, Parkinson's disease (PD) is the second most common form of neurodegenerative disease. Accumulation of a-synuclein in the brain (α-SYN) is a core feature of PD and leads to microglial activation, inflammatory cytokine/chemokine production, and ultimately to neurodegeneration. Given the importance of the JAK/STAT pathway in activating microglia and inducing cytokine/chemokine expression, JAK1 and/or TYK2 inhibitors may protect against neuroinflammation and neurodegeneration in PD (Qin et al. J Neurosci. 2016 May 4; 36(18):5144-59). Thus, brain penetrant JAK1 and/or TYK2 inhibitors may be able to treat neurodegenerative diseases such as amyotrophic lateral sclerosis (ALS), frontotemporal dementia (FTD), Alzheimer's disease, multiple sclerosis or Parkinson's disease (PD).

In some embodiments, the disease or disorder is cancer. In some embodiments, the cancer is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the cancer is lung adenocarcinoma, breast invasive carcinoma, uterine carcinosarcoma, ovarian serous cystadenocarcinoma, or stomach adenocarcinoma.

In some embodiments, the cancer is an adenocarcinoma, carcinoma, or cystadenocarcinoma.

In some embodiments, the cancer is uterine cancer, ovarian cancer, stomach cancer, esophageal cancer, lung cancer, bladder cancer, pancreatic cancer, or breast cancer.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}) Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” TYK2 and JAK1 kinases with a compound described herein includes the administration of a compound described herein to an individual or patient, such as a human, having TYK2 and JAK1 kinases, as well as, for example, introducing a compound described herein into a sample containing a cellular or purified preparation containing the TYK2 and JAK1 kinases.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of disorder or diseases associated with TYK2 and/or JAK1 as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

### Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure. Other agents such as therapeutic antibodies can be used in combination with the compounds of the present disclosure. The one or more additional pharmaceutical agents can be administered to a patient simultaneously or sequentially.

In some embodiments, the compound is administered or used in combination with a BCL2 inhibitor, a CDK2 inhibitor (e.g., INCB123666) or a CDK4/6 inhibitor (palbocitinib).

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Smll, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a compound of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, IHPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMIP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-a), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-IBB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating JAK1 and/or TYK2 in tissue samples, including human, and for identifying JAK1 and/or TYK2 binding of a labeled compound. Substitution of one or more of the atoms of the compounds of the present disclosure can also be useful in generating differentiated ADME (Adsorption, Distribution, Metabolism and Excretion.) Accordingly, the present disclosure includes JAK1 and/or TYK2 assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable isotopes that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃).

In some embodiments, alkyl groups of the disclosed Formulas (e.g., Formula (I)) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

Synthetic methods for including isotopes into organic compounds are known in the art (Deuterium Labeling in Organic Chemistry by Alan F. Thomas, New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro JAK1 and/or TYK2 labeling and competition assays, compounds that incorporate ³H, ¹⁴C, ⁸²Br, ¹²⁵I, ¹³¹I, or ³⁵S can be useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present disclosure can further include synthetic methods for incorporating radio-isotopes into compounds of the disclosure. Synthetic methods for incorporating radio-isotopes into organic compounds are well known in the art, and one of ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind and inhibit JAK1 and/or TYK2 by monitoring its concentration variation when contacting with JAK1 and/or TYK2, through tracking of the labeling. For example, a test compound (labeled) can be evaluated for its ability to reduce binding of another compound which is known to inhibit JAK1 and/or TYK2 (i.e., standard compound). Accordingly, the ability of a test compound to compete with the standard compound for binding to JAK1 and/or TYK2 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of JAK1 and/or TYK2-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). Typically, the flow rate used with the 30×100 mm column was 60 mL/minute.

pH=10 purifications: Waters XBridge C₁₈ 5 μm, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). Typically, the flow rate used with 30×100 mm column was 60 mL/minute.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1. 3-(8-((1r,4r)-4-(cyanomethyl)cyclohexyl)-7-methyl-8H-pyrrolo[2,3-d]thieno[3,2-b]pyridin-6-yl)-N,N-dimethylbenzamide

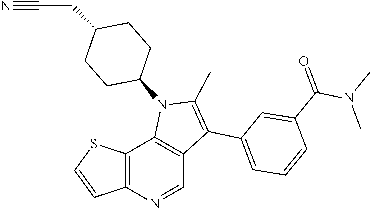 [SMILES: Cc1c(-c2cccc(C(=O)N(C)C)c2)c2cnc3ccsc3c2n1[C@H]1CC[C@H](CC#N)CC1]

### Step 1: 2-((1r,4r)-4-(thieno[3,2-b]pyridin-7-ylamino)cyclohexyl)acetonitrile

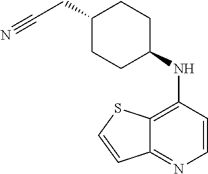 [SMILES: N#CC[C@H]1CC[C@H](Nc2ccnc3ccsc23)CC1]

To a screw-cap vial equipped with a magnetic stir bar was added 7-chlorothieno[3,2-b]pyridine (4.00 g, 23.58 mmol), dicyclohexyl(2′,6′-diisopropoxybiphenyl-2-yl)phosphine-(2′-aminobiphenyl-2-yl)(chloro)palladium (1:1) (2.77 g, 3.56 mmol), 2-((1r,4r)-4-aminocyclohexyl)acetonitrile, HCl salt (4.26 g, 24.39 mmol) and cesium carbonate (35.3 g, 108 mmol). The vial was sealed with a Teflon-lined septum, evacuated and backfilled with nitrogen (this process was repeated a total of three times). Anhydrous tBuOH (200.0 mL) was added via syringe. The mixture was stirred at 85° C. for 16 h. After cooling to room temperature, the mixture was filtered and concentrated. The residue was purified on silica gel (120 g, 0-100% EtOAc in CH₂Cl₂, then 15% MeOH in CH₂Cl₂) to give the desired product as a yellow foamy solid (4.66 g, 73%). LC-MS calculated for C₁₅H₁₈N₃S (M+H)⁺ m/z=272.1; found 272.1.

### Step 2: 2-((1r,4r)-4-((6-iodothieno[3,2-b]pyridin-7-yl)amino)cyclohexyl)acetonitrile

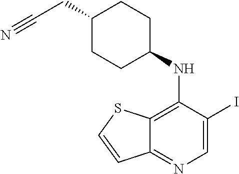 [SMILES: N#CC[C@H]1CC[C@H](Nc2c(I)cnc3ccsc23)CC1]

To a mixture of 2-((1r,4r)-4-(thieno[3,2-b]pyridin-7-ylamino)cyclohexyl)acetonitrile (4.66 g, 17.17 mmol) and 1-iodopyrrolidine-2,5-dione (3.926 g, 17.45 mmol) was added acetonitrile (100.0 mL). The mixture was heated to 90° C. for 20 h. After cooling to room temperature, the mixture was concentrated. The residue was dissolved in CH₂Cl₂ and washed with 2M K₂CO₃ (aq). The separated organic layer was dried over Na₂SO₄, filtered and concentrated. The residue was purified on silica gel (120 g, 0-100% EtOAc in DCM) to give the desired product as a yellow foamy solid (4.45 g, 65%). LC-MS calculated for C₁₅H₁₇IN₃S (M+H)⁺ m/z=398.0; found 398.1.

### Step 3: 2-((1r,4r)-4-(7-methyl-8H-pyrrolo[2,3-d]thieno[3,2-b]pyridin-8-yl)cyclohexyl)acetonitrile

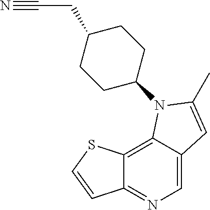 [SMILES: Cc1cc2cnc3ccsc3c2n1[C@H]1CC[C@H](CC#N)CC1]

To a screw-cap vial equipped with a magnetic stir bar was added 2-((1r,4r)-4-((6-iodothieno[3,2-b]pyridin-7-yl)amino)cyclohexyl)acetonitrile (4.45 g, 11.20 mmol), palladium(II) acetate (691.2 mg, 3.08 mmol), lithium chloride (799.9 mg, 18.87 mmol) and K₂CO₃ (7.937 g, 57.4 mmol). The vial was sealed with a Teflon-lined septum, evacuated and backfilled with nitrogen (this process was repeated a total of three times). A solution of allyl acetate (3.52 g, 35.2 mmol) in DMF (100 mL) was added via syringe. The mixture was stirred at 120° C. for 20 h. After cooling to room temperature, the reaction mixture was filtered. The filtrate was concentrated. The residue was purified on silica gel (40 g, 0-100% EtOAc in CH₂Cl₂) to give the desired product (1093 mg, 32%). LC-MS calculated for C₁₈H₂₀N₃S (M+H)⁺ m/z=310.1; found 310.1.

### Step 4: 2-((1r,4r)-4-(6-bromo-7-methyl-8H-pyrrolo[2,3-d]thieno[3,2-b]pyridin-8-yl)cyclohexyl)acetonitrile

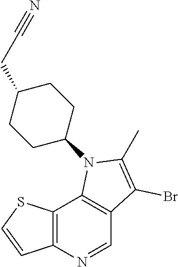 [SMILES: Cc1c(Br)c2cnc3ccsc3c2n1[C@H]1CC[C@H](CC#N)CC1]

To a mixture of 2-((1r,4r)-4-(7-methyl-8H-pyrrolo[2,3-d]thieno[3,2-b]pyridin-8-yl)cyclohexyl)acetonitrile (512.5 mg, 1.656 mmol) and 1-bromopyrrolidine-2,5-dione (335.3 mg, 1.884 mmol) was added CH₂Cl₂ (10.0 mL) followed by DMF (10.00 mL). The mixture was stirred at room temperature for 2 h. The reaction mixture was concentrated. The residue was purified on silica gel (20 g, 0-100% EtOAc in CH₂Cl₂) to give the desired product as a white solid (398.8 mg, 62%). LC-MS calculated for C₁₈H₁₉BrN₃S (M+H)⁺ m/z=388.0; found 388.1.

### Step 5: 3-(8-((1r,4r)-4-(cyanomethyl)cyclohexyl)-7-methyl-8H-pyrrolo[2,3-d]thieno[3,2-b]pyridin-6-yl)-N,N-dimethylbenzamide

To a screw-cap vial equipped with a magnetic stir bar was added 2-((1r,4r)-4-(6-bromo-7-methyl-8H-pyrrolo[2,3-d]thieno[3,2-b]pyridin-8-yl)cyclohexyl)acetonitrile (15.0 mg, 0.039 mmol), N,N-dimethyl-3-(4,4,5,5-tetramethyl-1,3,2-dioxaborolan-2-yl)benzamide (19.0 mg, 0.069 mmol), dicyclohexyl(2′,4′,6′-triisopropylbiphenyl-2-yl)phosphine-(2′-aminobiphenyl-2-yl)(chloro)palladium (1:1) (4.6 mg, 5.85 μmol) and cesium carbonate (45.9 mg, 0.141 mmol). The vial was sealed with a Teflon-lined septum, evacuated and backfilled with nitrogen (this process was repeated a total of three times). 1,4-Dioxane (2.0 mL) was added via syringe, followed by degassed water (200.0 μL). After stirring at 80° C. for 16 h, the reaction was cooled to room temperature. MeOH (2.0 mL) was added followed by HCl (4.0 M in dioxane) (1.0 mL, 4.00 mmol). After stirring at room temperature for 30 min, the reaction mixture was filtered. The filtrate was purified using prep-LCMS (XBridge C18 column, eluting with a gradient of acetonitrile/water containing 0.1% TFA, at flow rate of 60 mL/min) to afford the desired product as its TFA salt. LC-MS calculated for C₂₇H₂₉N₄OS (M+H)⁺: m/z=457.2; found: 457.2.

### Example 2. 2-((1r,4r)-4-(7-methyl-6-(3-(pyrrolidine-1-carbonyl)phenyl)-8H-pyrrolo[2,3-d]thieno[3,2-b]pyridin-8-yl)cyclohexyl)acetonitrile

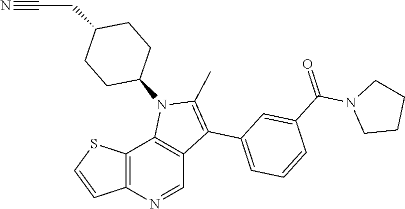 [SMILES: Cc1c(-c2cccc(C(=O)N3CCCC3)c2)c2cnc3ccsc3c2n1[C@H]1CC[C@H](CC#N)CC1]

This compound was prepared according to the procedure described in Example 1, using (3-(pyrrolidine-1-carbonyl)phenyl)boronic acid instead of N,N-dimethyl-3-(4,4,5,5-tetramethyl-1,3,2-dioxaborolan-2-yl)benzamide as the starting material. LC-MS calculated for C₂₉H₃₁N₄OS (M+H)⁺: m/z=483.2; found 483.2.

### Example 3. 2-((1r,4r)-4-(7-methyl-6-(3-oxoisoindolin-5-yl)-8H-pyrrolo[2,3-d]thieno[3,2-b]pyridin-8-yl)cyclohexyl)acetonitrile

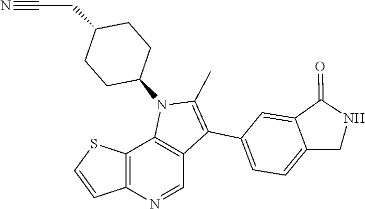 [SMILES: Cc1c(-c2ccc3c(c2)C(=O)NC3)c2cnc3ccsc3c2n1[C@H]1CC[C@H](CC#N)CC1]

This compound was prepared according to the procedure described in Example 1, using 6-(4,4,5,5-tetramethyl-1,3,2-dioxaborolan-2-yl)isoindolin-1-one instead of N,N-dimethyl-3-(4,4,5,5-tetramethyl-1,3,2-dioxaborolan-2-yl)benzamide as the starting material. LC-MS calculated for C₂₆H₂₅N₄OS (M+H)⁺: m/z=441.2; found 441.2.

### Example 4. N-(3-(8-((1r,4r)-4-(cyanomethyl)cyclohexyl)-7-methyl-8H-pyrrolo[2,3-d]thieno[3,2-b]pyridin-6-yl)phenyl)acetamide

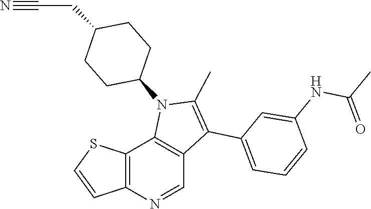 [SMILES: CC(=O)Nc1cccc(-c2c(C)n([C@H]3CC[C@H](CC#N)CC3)c3c2cnc2ccsc23)c1]

This compound was prepared according to the procedure described in Example 1, using (3-acetamidophenyl)boronic acid instead of N,N-dimethyl-3-(4,4,5,5-tetramethyl-1,3,2-dioxaborolan-2-yl)benzamide as the starting material. LC-MS calculated for C₂₆H₂₇N₄OS (M+H)⁺: m/z=443.2; found 443.2.

### Example 5. 4-(8-((1r,4r)-4-(cyanomethyl)cyclohexyl)-7-methyl-8H-pyrrolo[2,3-d]thieno[3,2-b]pyridin-6-yl)-N,N-dimethylpicolinamide

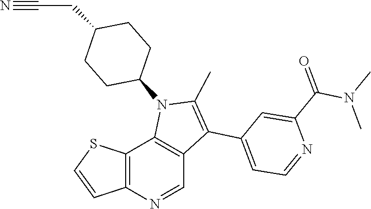 [SMILES: Cc1c(-c2ccnc(C(=O)N(C)C)c2)c2cnc3ccsc3c2n1[C@H]1CC[C@H](CC#N)CC1]

This compound was prepared according to the procedure described in Example 1, using N,N-dimethyl-4-(4,4,5,5-tetramethyl-1,3,2-dioxaborolan-2-yl)picolinamide instead of N,N-dimethyl-3-(4,4,5,5-tetramethyl-1,3,2-dioxaborolan-2-yl)benzamide as the starting material. LC-MS calculated for C₂₆H₂₈N₅OS (M+H)⁺: m/z=458.2; found 458.1.

### Example 6. 2-((1r,4r)-4-(7-methyl-6-(2-(4-methylpiperazin-1-yl)pyridin-4-yl)-8H-pyrrolo[2,3-d]thieno[3,2-b]pyridin-8-yl)cyclohexyl)acetonitrile

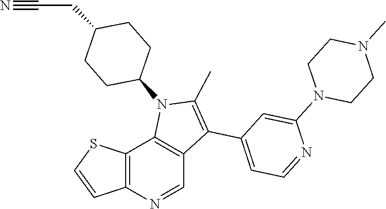 [SMILES: Cc1c(-c2ccnc(N3CCN(C)CC3)c2)c2cnc3ccsc3c2n1[C@H]1CC[C@H](CC#N)CC1]

This compound was prepared according to the procedure described in Example 1, using 1-methyl-4-(4-(4,4,5,5-tetramethyl-1,3,2-dioxaborolan-2-yl)pyridin-2-yl)piperazine instead of N,N-dimethyl-3-(4,4,5,5-tetramethyl-1,3,2-dioxaborolan-2-yl)benzamide as the starting material. LC-MS calculated for C₂₈H₃₃N₆S (M+H)⁺: m/z=485.2; found 485.2.

### Example 7. 2-((1r,4r)-4-(6-(4-fluoro-3-(pyrrolidin-1-ylmethyl)phenyl)-7-methyl-8H-pyrrolo[2,3-d]thieno[3,2-b]pyridin-8-yl)cyclohexyl)acetonitrile

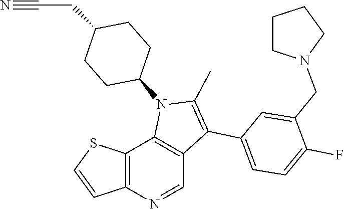 [SMILES: Cc1c(-c2ccc(F)c(CN3CCCC3)c2)c2cnc3ccsc3c2n1[C@H]1CC[C@H](CC#N)CC1]

This compound was prepared according to the procedure described in Example 1, using 1-(2-fluoro-5-(4,4,5,5-tetramethyl-1,3,2-dioxaborolan-2-yl)benzyl)pyrrolidine instead of N,N-dimethyl-3-(4,4,5,5-tetramethyl-1,3,2-dioxaborolan-2-yl)benzamide as the starting material. LC-MS calculated for C₂₉H₃₂FN₄S (M+H)⁺: m/z=487.2; found 487.2.

### Example 8. 5-(8-((1r,4r)-4-(cyanomethyl)cyclohexyl)-7-methyl-8H-pyrrolo[2,3-d]thieno[3,2-b]pyridin-6-yl)-2-fluoro-N,N-dimethylbenzamide

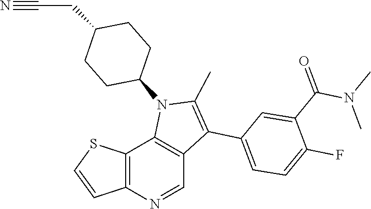 [SMILES: Cc1c(-c2ccc(F)c(C(=O)N(C)C)c2)c2cnc3ccsc3c2n1[C@H]1CC[C@H](CC#N)CC1]

This compound was prepared according to the procedure described in Example 1, using (3-(dimethylcarbamoyl)-4-fluorophenyl)boronic acid instead of N,N-dimethyl-3-(4,4,5,5-tetramethyl-1,3,2-dioxaborolan-2-yl)benzamide as the starting material. LC-MS calculated for C₂₇H₂₈FN₄OS (M+H)⁺: m/z=475.2; found 475.2.

### Example 9. 4-(8-((1r,4r)-4-(cyanomethyl)cyclohexyl)-7-methyl-8H-pyrrolo[2,3-d]thieno[3,2-b]pyridin-6-yl)-N,N-dimethylbenzamide

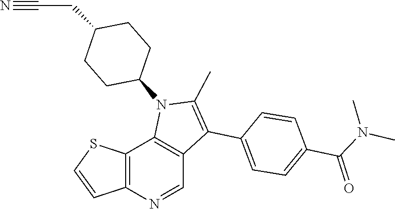 [SMILES: Cc1c(-c2ccc(C(=O)N(C)C)cc2)c2cnc3ccsc3c2n1[C@H]1CC[C@H](CC#N)CC1]

This compound was prepared according to the procedure described in Example 1, using N,N-dimethyl-4-(4,4,5,5-tetramethyl-1,3,2-dioxaborolan-2-yl)benzamide instead of N,N-dimethyl-3-(4,4,5,5-tetramethyl-1,3,2-dioxaborolan-2-yl)benzamide as the starting material. LC-MS calculated for C₂₇H₂₉N₄OS (M+H)⁺: m/z=457.2; found 457.2.

### Example 10. 4-(8-((1r,4r)-4-(cyanomethyl)cyclohexyl)-7-methyl-8H-pyrrolo[2,3-d]thieno[3,2-b]pyridin-6-yl)-3-fluoro-N,N-dimethylbenzamide

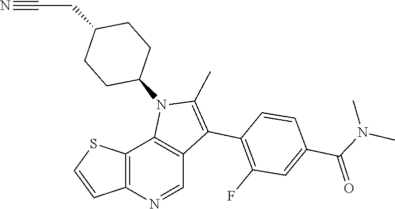 [SMILES: Cc1c(-c2ccc(C(=O)N(C)C)cc2F)c2cnc3ccsc3c2n1[C@H]1CC[C@H](CC#N)CC1]

This compound was prepared according to the procedure described in Example 1, using (4-(dimethylcarbamoyl)-2-fluorophenyl)boronic acid instead of N,N-dimethyl-3-(4,4,5,5-tetramethyl-1,3,2-dioxaborolan-2-yl)benzamide as the starting material. LC-MS calculated for C₂₇H₂₈FN₄OS (M+H)⁺: m/z=475.2; found 475.2.

### Example 11. 4-(8-((1r,4r)-4-(cyanomethyl)cyclohexyl)-7-methyl-8H-pyrrolo[2,3-d]thieno[3,2-b]pyridin-6-yl)-2-fluoro-N,N-dimethylbenzamide

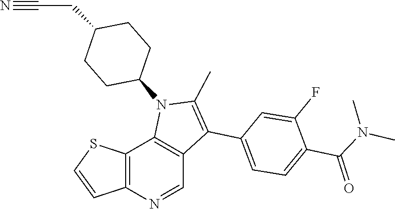 [SMILES: Cc1c(-c2ccc(C(=O)N(C)C)c(F)c2)c2cnc3ccsc3c2n1[C@H]1CC[C@H](CC#N)CC1]

This compound was prepared according to the procedure described in Example 1, using (4-(dimethylcarbamoyl)-3-fluorophenyl)boronic acid instead of N,N-dimethyl-3-(4,4,5,5-tetramethyl-1,3,2-dioxaborolan-2-yl)benzamide as the starting material. LC-MS calculated for C₂₇H₂₈FN₄OS (M+H)⁺: m/z=475.2; found 475.1.

### Example 12. 5-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-2-carboxamide

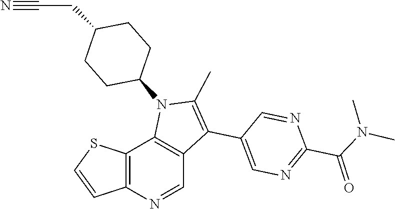 [SMILES: Cc1c(-c2cnc(C(=O)N(C)C)nc2)c2cnc3ccsc3c2n1[C@H]1CC[C@H](CC#N)CC1]

This compound was prepared according to the procedure described in Example 1, using N,N-dimethyl-5-(4,4,5,5-tetramethyl-1,3,2-dioxaborolan-2-yl)pyrimidine-2-carboxamide instead of N,N-dimethyl-3-(4,4,5,5-tetramethyl-1,3,2-dioxaborolan-2-yl)benzamide as the starting material. LC-MS calculated for C₂₅H₂₇N₆OS (M+H)⁺: m/z=459.2; found 459.2.

### Example 13. 5-(8-((1r,4r)-4-(cyanomethyl)cyclohexyl)-7-methyl-8H-pyrrolo[2,3-d]thieno[3,2-b]pyridin-6-yl)pyrimidine-2-carbonitrile

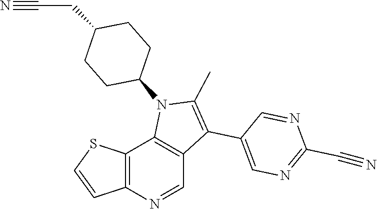 [SMILES: Cc1c(-c2cnc(C#N)nc2)c2cnc3ccsc3c2n1[C@H]1CC[C@H](CC#N)CC1]

This compound was prepared according to the procedure described in Example 1, using 5-(4,4,5,5-tetramethyl-1,3,2-dioxaborolan-2-yl)pyrimidine-2-carbonitrile instead of N,N-dimethyl-3-(4,4,5,5-tetramethyl-1,3,2-dioxaborolan-2-yl)benzamide as the starting material. LC-MS calculated for C₂₃H₂₁N₆S (M+H)⁺: m/z=413.2; found 413.1.

### Example 14. 2-((1r,4r)-4-(6-(1-isopropyl-1H-pyrazol-4-yl)-7-methyl-8H-pyrrolo[2,3-d]thieno[3,2-b]pyridin-8-yl)cyclohexyl)acetonitrile

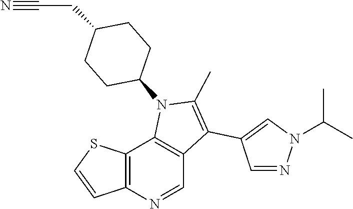 [SMILES: Cc1c(-c2cnn(C(C)C)c2)c2cnc3ccsc3c2n1[C@H]1CC[C@H](CC#N)CC1]

This compound was prepared according to the procedure described in Example 1, using 1-isopropyl-4-(4,4,5,5-tetramethyl-1,3,2-dioxaborolan-2-yl)-1H-pyrazole instead of N,N-dimethyl-3-(4,4,5,5-tetramethyl-1,3,2-dioxaborolan-2-yl)benzamide as the starting material. LC-MS calculated for C₂₄H₂₈N₅S (M+H)⁺: m/z=418.2; found 418.2.

### Example 15. 2-((1r,4r)-4-(7-methyl-6-(1-(tetrahydro-2H-pyran-4-yl)-1H-pyrazol-4-yl)-8H-pyrrolo[2,3-d]thieno[3,2-b]pyridin-8-yl)cyclohexyl)acetonitrile

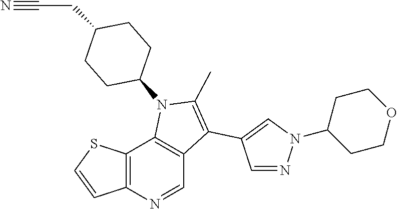 [SMILES: Cc1c(-c2cnn(C3CCOCC3)c2)c2cnc3ccsc3c2n1[C@H]1CC[C@H](CC#N)CC1]

This compound was prepared according to the procedure described in Example 1, using 1-(tetrahydro-2H-pyran-4-yl)-4-(4,4,5,5-tetramethyl-1,3,2-dioxaborolan-2-yl)-1H-pyrazole instead of N,N-dimethyl-3-(4,4,5,5-tetramethyl-1,3,2-dioxaborolan-2-yl)benzamide as the starting material. LC-MS calculated for C₂₆H₃₀N₅OS (M+H)⁺: m/z=460.2; found 460.2.

### Example 16. 2-((1r,4r)-4-(6-(4-(1-aminocyclopropyl)phenyl)-7-methyl-8H-pyrrolo[2,3-d]thieno[3,2-b]pyridin-8-yl)cyclohexyl)acetonitrile

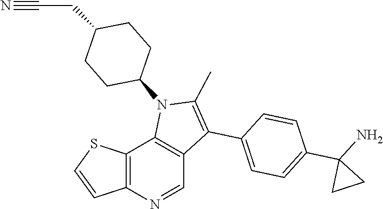 [SMILES: Cc1c(-c2ccc(C3(N)CC3)cc2)c2cnc3ccsc3c2n1[C@H]1CC[C@H](CC#N)CC1]

This compound was prepared according to the procedure described in Example 1, using (4-(1-((tert-butoxycarbonyl)amino)cyclopropyl)phenyl)boronic acid instead of N,N-dimethyl-3-(4,4,5,5-tetramethyl-1,3,2-dioxaborolan-2-yl)benzamide as the starting material. LC-MS calculated for C₂₇H₂₉N₄S (M+H)⁺: m/z=441.2; found 441.2.

### Example 17. 2-((1r,4r)-4-(6-(4-(1-aminocyclobutyl)phenyl)-7-methyl-8H-pyrrolo[2,3-d]thieno[3,2-b]pyridin-8-yl)cyclohexyl)acetonitrile

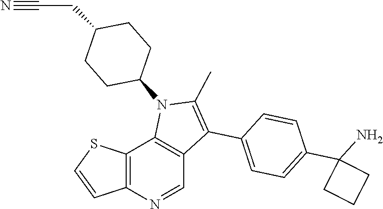 [SMILES: Cc1c(-c2ccc(C3(N)CCC3)cc2)c2cnc3ccsc3c2n1[C@H]1CC[C@H](CC#N)CC1]

This compound was prepared according to the procedure described in Example 1, using tert-butyl (1-(4-(4,4,5,5-tetramethyl-1,3,2-dioxaborolan-2-yl)phenyl)cyclobutyl)carbamate instead of N,N-dimethyl-3-(4,4,5,5-tetramethyl-1,3,2-dioxaborolan-2-yl)benzamide as the starting material. LC-MS calculated for C₂₈H₃₁N₄S (M+H)⁺: m/z=455.2; found 455.3.

### Example 18. 2-((1r,4r)-4-(7-methyl-6-(pyrimidin-2-yl)-8H-pyrrolo[2,3-d]thieno[3,2-b]pyridin-8-yl)cyclohexyl)acetonitrile

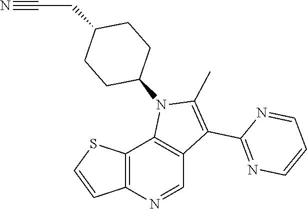 [SMILES: Cc1c(-c2ncccn2)c2cnc3ccsc3c2n1[C@H]1CC[C@H](CC#N)CC1]

To a screw-cap vial equipped with a magnetic stir bar was added 2-((1r,4r)-4-(6-bromo-7-methyl-8H-pyrrolo[2,3-d]thieno[3,2-b]pyridin-8-yl)cyclohexyl)acetonitrile (20.6 mg, 0.053 mmol), 2-dicyclohexylphosphino-2′,4′,6′-triisopropylbiphenyl (10.1 mg, 0.021 mmol), CsF (55.1 mg, 0.363 mmol) and palladium(II) acetate (2.0 mg, 8.91 μmol). The vial was sealed with a Teflon-lined septum, evacuated and backfilled with nitrogen (this process was repeated a total of three times). A solution of 2-(tributylstannyl)pyrimidine (68.4 mg, 0.185 mmol) in 1,4-dioxane (3.0 mL) was added via syringe. After stirring at 80° C. for 16 h, the reaction was cooled to room temperature. MeOH (2.0 mL) was added followed by HCl (4.0 M in dioxane) (1.0 mL, 4.00 mmol). After stirring at room temperature for 30 min, the reaction mixture was filtered. The filtrate was purified using prep-LCMS (XBridge C18 column, eluting with a gradient of acetonitrile/water containing 0.1% TFA, at flow rate of 60 mL/min) to afford the desired product as its TFA salt. LC-MS calculated for C₂₂H₂₂N₅S (M+H)⁺: m/z=388.2; found: 388.1.

### Example 19. 2-((1r,4r)-4-(7-methyl-6-(pyridazin-4-yl)-8H-pyrrolo[2,3-d]thieno[3,2-b]pyridin-8-yl)cyclohexyl)acetonitrile

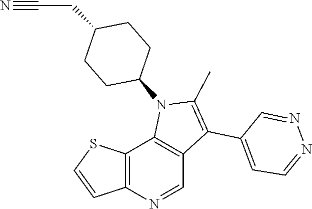 [SMILES: Cc1c(-c2ccnnc2)c2cnc3ccsc3c2n1[C@H]1CC[C@H](CC#N)CC1]

This compound was prepared according to the procedure described in Example 18, using 4-(tributylstannyl)pyridazine instead of 2-(tributylstannyl)pyrimidine as the starting material. LC-MS calculated for C₂₂H₂₂N₅S (M+H)⁺: m/z=388.2; found: 388.2.

### Example 20. 2-((1r,4r)-4-(7-methyl-6-(pyrazin-2-yl)-8H-pyrrolo[2,3-d]thieno[3,2-b]pyridin-8-yl)cyclohexyl)acetonitrile

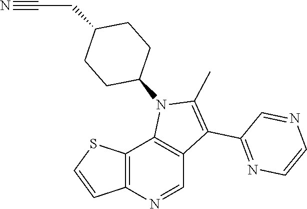 [SMILES: Cc1c(-c2cnccn2)c2cnc3ccsc3c2n1[C@H]1CC[C@H](CC#N)CC1]

This compound was prepared according to the procedure described in Example 18, using 2-(tributylstannyl)pyrazine instead of 2-(tributylstannyl)pyrimidine as the starting material. LC-MS calculated for C₂₂H₂₂N₅S (M+H)⁺: m/z=388.2; found: 388.2.

### Example 21. 2-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-4-carboxamide

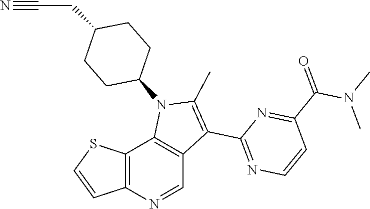 [SMILES: Cc1c(-c2nccc(C(=O)N(C)C)n2)c2cnc3ccsc3c2n1[C@H]1CC[C@H](CC#N)CC1]

### Step 1: 2-chloro-N,N-dimethylpyrimidine-4-carboxamide

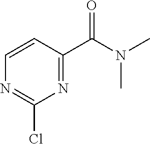

To a mixture of 2-chloropyrimidine-4-carboxylic acid (511.6 mg, 3.23 mmol) and N,N,N′,N′-tetramethyl-O-(7-azabenzotriazol-1-yl)uronium hexafluorophosphate (3.77 g, 9.92 mmol) was added DMF (20.0 mL) followed by dimethylamine (2.0 M in THF) (3.8 mL, 7.60 mmol). The mixture was stirred at room temperature for 2 h. The reaction was diluted with CH₂Cl₂ and washed with 2M K₂CO₃ (aq). The separated organic layer was dried over anhydrous Na₂SO₄, filtered and concentrated. The residue was purified on silica gel (20 g, 0-100% EtOAc in CH₂Cl₂) to give the desired product as a white solid (304.6 mg, 51%). LC-MS calculated for C₇H₉ClN₃O (M+H)⁺: m/z=186.0; found: 186.0.

### Step 2: N,N-dimethyl-2-(tributylstannyl)pyrimidine-4-carboxamide

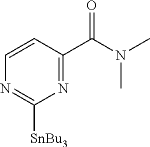

To a screw-cap vial equipped with a magnetic stir bar was added 2-chloro-N,N-dimethylpyrimidine-4-carboxamide (304.6 mg, 1.641 mmol) and bis(di-tert-butyl(4-dimethylaminophenyl)phosphine)dichloropalladium(II) (350.3 mg, 0.495 mmol). The vial was sealed with a Teflon-lined septum, evacuated and backfilled with nitrogen (this process was repeated a total of three times). A solution of bis(tributyltin) (1965 mg, 3.39 mmol) in 1,4-dioxane (15.0 mL) was added via syringe. After stirring at 130° C. for 16 h, the reaction was cooled to room temperature and concentrated. The residue was purified on silica gel (20 g, 0-100% EtOAc in CH₂Cl₂) to give the desired product as a colorless oil (406.8 mg, 56%). LC-MS calculated for C₁₉H₃₆N₃OSn (M+H)⁺: m/z=442.2; found: 442.2.

### Step 3: 2-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-4-carboxamide

To a screw-cap vial equipped with a magnetic stir bar was added 2-((1r,4r)-4-(6-bromo-7-methyl-8H-pyrrolo[2,3-d]thieno[3,2-b]pyridin-8-yl)cyclohexyl)acetonitrile (22.0 mg, 0.057 mmol), 2-dicyclohexylphosphino-2′,4′,6′-triisopropylbiphenyl (10.8 mg, 0.023 mmol), CsF (57.5 mg, 0.379 mmol) and palladium(II) acetate (2.6 mg, 0.012 mmol). The vial was sealed with a Teflon-lined septum, evacuated and backfilled with nitrogen (this process was repeated a total of three times). A solution of N,N-dimethyl-2-(tributylstannyl)pyrimidine-4-carboxamide (69.6 mg, 0.158 mmol) in 1,4-dioxane (3.0 mL) was added via syringe. After stirring at 80° C. for 16 h, the reaction was cooled to room temperature. MeOH (2.0 mL) was added followed by HCl (4.0 M in dioxane) (1.0 mL, 4.00 mmol). After stirring at room temperature for 30 min, the reaction mixture was filtered. The filtrate was purified using prep-LCMS (XBridge C18 column, eluting with a gradient of acetonitrile/water containing 0.1% TFA, at flow rate of 60 mL/min) to afford the desired product as its TFA salt. LC-MS calculated for C₂₅H₂₇N₆OS (M+H)⁺: m/z=459.2; found: 459.2.

### Example 22. 2-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-5-carboxamide

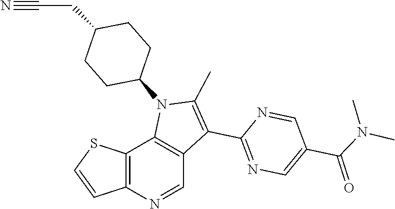 [SMILES: Cc1c(-c2ncc(C(=O)N(C)C)cn2)c2cnc3ccsc3c2n1[C@H]1CC[C@H](CC#N)CC1]

This compound was prepared according to the procedure described in Example 21, using 2-chloropyrimidine-5-carboxylic acid instead of 2-chloropyrimidine-4-carboxylic acid as the starting material. LC-MS calculated for C₂₅H₂₇N₆OS (M+H)⁺: m/z=459.2; found: 459.2.

### Example 23. 2-((1r,4r)-4-(6-(3-(6-oxa-2-azaspiro[3.4]octane-2-carbonyl)phenyl)-7-methyl-8H-pyrrolo[2,3-d]thieno[3,2-b]pyridin-8-yl)cyclohexyl)acetonitrile

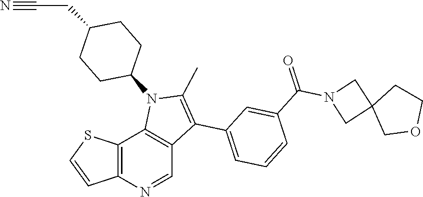 [SMILES: Cc1c(-c2cccc(C(=O)N3CC4(CCOC4)C3)c2)c2cnc3ccsc3c2n1[C@H]1CC[C@H](CC#N)CC1]

### Step 1: methyl 3-(8-((1r,4r)-4-(cyanomethyl)cyclohexyl)-7-methyl-8H-pyrrolo[2,3-d]thieno[3,2-b]pyridin-6-yl)benzoate

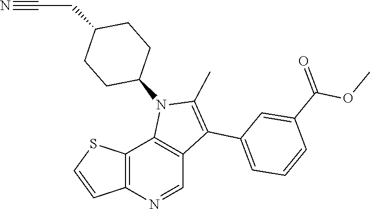 [SMILES: COC(=O)c1cccc(-c2c(C)n([C@H]3CC[C@H](CC#N)CC3)c3c2cnc2ccsc23)c1]

A mixture of 2-((1r,4r)-4-(6-bromo-7-methyl-8H-pyrrolo[2,3-d]thieno[3,2-b]pyridin-8-yl)cyclohexyl)acetonitrile (see step 4 in example 1, 430 mg, 1.107 mmol), (3-(methoxycarbonyl)phenyl)boronic acid (399 mg, 2.215 mmol), dicyclohexyl(2′,4′,6′-triisopropylbiphenyl-2-yl)phosphine-(2′-aminobiphenyl-2-yl)(chloro)palladium (1:1) (83 mg, 0.105 mmol) and cesium carbonate (722 mg, 2.215 mmol) was evacuated and backfilled with nitrogen (this process was repeated a total of three times). 1,4-Dioxane (8.0 mL) was added, followed by degassed Water (200.0 μL, 11.10 mmol). The reaction was heated at 70° C. for 2 h. After cooling to room temperature, the mixture was purified by silica gel column (0-100% EtOAc/Hexanes) to provide methyl 3-(8-((1r,4r)-4-(cyanomethyl)cyclohexyl)-7-methyl-8H-pyrrolo[2,3-d]thieno[3,2-b]pyridin-6-yl)benzoate (270 mg, 0.609 mmol, 55.0% yield). LC-MS calculated for C₂₆H₂₆N₃O₂S (M+H)⁺ m/z=444.2; found 444.1.

### Step 2: 3-(8-((1r,4r)-4-(cyanomethyl)cyclohexyl)-7-methyl-8H-pyrrolo[2,3-d]thieno[3,2-b]pyridin-6-yl)benzoic acid

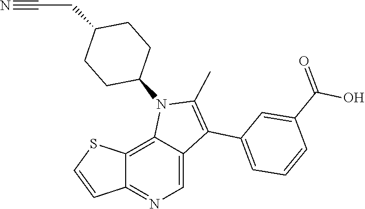 [SMILES: Cc1c(-c2cccc(C(=O)O)c2)c2cnc3ccsc3c2n1[C@H]1CC[C@H](CC#N)CC1]

A mixture of methyl 3-(8-((1r,4r)-4-(cyanomethyl)cyclohexyl)-7-methyl-8H-pyrrolo[2,3-d]thieno[3,2-b]pyridin-6-yl)benzoate (270 mg, 0.609 mmol) in MeOH (10 mL) was treated with a solution of lithium hydride (58.1 mg, 7.30 mmol) in Water (4 mL, 222 mmol) at 45° C. overnight. The mixture was concentrated, diluted with DCM, neutralized with aq. HCl solution. The organic layer was dried over MgSO₄ and concentrated to provide 3-(8-((1r,4r)-4-(cyanomethyl)cyclohexyl)-7-methyl-8H-pyrrolo[2,3-d]thieno[3,2-b]pyridin-6-yl)benzoic acid (96 mg, 0.223 mmol, 36.7% yield). LC-MS calculated for C₂₅H₂₄N₃O₂S (M+H)⁺ m/z=430.2; found 430.1.

### Step 3: 2-((1r,4r)-4-(6-(3-(6-oxa-2-azaspiro[3.4]octane-2-carbonyl)phenyl)-7-methyl-8H-pyrrolo[2,3-d]thieno[3,2-b]pyridin-8-yl)cyclohexyl)acetonitrile

A mixture of 3-(8-((1r,4r)-4-(cyanomethyl)cyclohexyl)-7-methyl-8H-pyrrolo[2,3-d]thieno[3,2-b]pyridin-6-yl)benzoic acid (4 mg, 9.31 μmol), 6-oxa-2-azaspiro[3.4]octane (1.054 mg, 9.31 μmol), HATU (3.54 mg, 9.31 μmol) and hunig's base (3.25 μl, 0.019 mmol) in DMF (0.5 mL) was stirred at room temperature for 2 h. The mixture was purified by prep-LCMS (XBridge C18 column, eluting with a gradient of acetonitrile/water containing 0.1% TFA, at flow rate of 60 mL/min) to provide 2-((1r,4r)-4-(6-(3-(6-oxa-2-azaspiro[3.4]octane-2-carbonyl)phenyl)-7-methyl-8H-pyrrolo[2,3-d]thieno[3,2-b]pyridin-8-yl)cyclohexyl)acetonitrile (1.4 mg, 2.67 μmol, 28.7% yield) as its TFA salt. LC-MS calculated for C₃₁H₃₃N₄O₂S (M+H)⁺ m/z=525.2; found 525.1.

### Example 24. 2-((1r,4r)-4-(7-methyl-6-(3-(7-oxo-2,6-diazaspiro[3.4]octane-2-carbonyl)phenyl)-8H-pyrrolo[2,3-d]thieno[3,2-b]pyridin-8-yl)cyclohexyl)acetonitrile

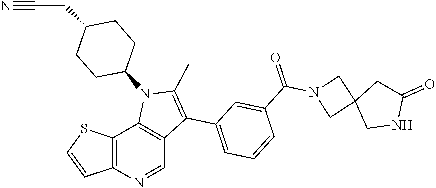 [SMILES: Cc1c(-c2cccc(C(=O)N3CC4(CNC(=O)C4)C3)c2)c2cnc3ccsc3c2n1[C@H]1CC[C@H](CC#N)CC1]

This compound was prepared according to the procedure described in Example 23, using 2,6-diazaspiro[3.4]octan-7-one as the starting material. LC-MS calculated for C₃₁H₃₂N₅O₂S (M+H)⁺: m/z=538.2; found 538.2.

### Example 25. 2-((1r,4r)-4-(6-(3-(3-hydroxy-3-methylazetidine-1-carbonyl)phenyl)-7-methyl-8H-pyrrolo[2,3-d]thieno[3,2-b]pyridin-8-yl)cyclohexyl)acetonitrile

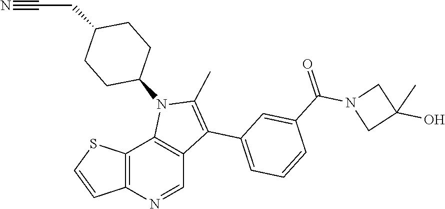 [SMILES: Cc1c(-c2cccc(C(=O)N3CC(C)(O)C3)c2)c2cnc3ccsc3c2n1[C@H]1CC[C@H](CC#N)CC1]

This compound was prepared according to the procedure described in Example 23, using 3-methylazetidin-3-ol, HCl salt as the starting material. LC-MS calculated for C₂₉H₃₁N₄O₂S (M+H)⁺: m/z=499.2; found 499.2.

### Example 26. 2-((1R,4r)-4-(6-(3-((R)-3-methoxypyrrolidine-1-carbonyl)phenyl)-7-methyl-8H-pyrrolo[2,3-d]thieno[3,2-b]pyridin-8-yl)cyclohexyl)acetonitrile

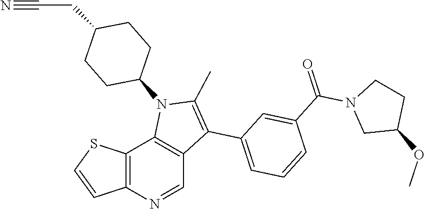 [SMILES: CO[C@@H]1CCN(C(=O)c2cccc(-c3c(C)n([C@H]4CC[C@H](CC#N)CC4)c4c3cnc3ccsc34)c2)C1]

This compound was prepared according to the procedure described in Example 23, using (R)-3-methoxypyrrolidine as the starting material. LC-MS calculated for C₃₀H₃₃N₄O₂S (M+H)⁺: m/z=513.2; found 513.2.

### Example 27. 2-((1r,4r)-4-(7-methyl-6-(3-(morpholine-4-carbonyl)phenyl)-8H-pyrrolo[2,3-d]thieno[3,2-b]pyridin-8-yl)cyclohexyl)acetonitrile

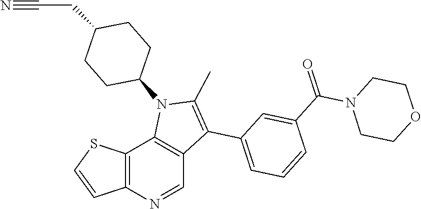 [SMILES: Cc1c(-c2cccc(C(=O)N3CCOCC3)c2)c2cnc3ccsc3c2n1[C@H]1CC[C@H](CC#N)CC1]

This compound was prepared according to the procedure described in Example 23, using morpholine as the starting material. LC-MS calculated for C₂₉H₃₁N₄O₂S (M+H)⁺: m/z=499.2; found 499.2.

### Example 28. 2-((1r,4r)-4-(7-methyl-6-(3-(4-methylpiperazine-1-carbonyl)phenyl)-8H-pyrrolo[2,3-d]thieno[3,2-b]pyridin-8-yl)cyclohexyl)acetonitrile

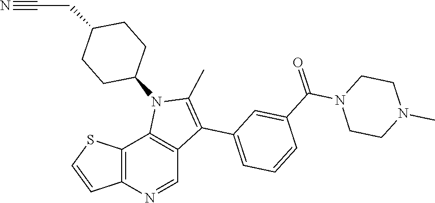 [SMILES: Cc1c(-c2cccc(C(=O)N3CCN(C)CC3)c2)c2cnc3ccsc3c2n1[C@H]1CC[C@H](CC#N)CC1]

This compound was prepared according to the procedure described in Example 23, using 1-methylpiperazine as the starting material. LC-MS calculated for C₃₀H₃₄N₅OS (M+H)⁺: m/z=512.2; found 512.2.

### Example 29. 3-(8-((1r,4r)-4-(cyanomethyl)cyclohexyl)-7-(2-hydroxyethyl)-8H-pyrrolo[2,3-d]thieno[3,2-b]pyridin-6-yl)-N,N-dimethylbenzamide

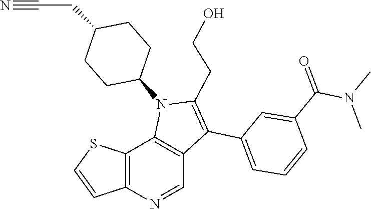 [SMILES: CN(C)C(=O)c1cccc(-c2c(CCO)n([C@H]3CC[C@H](CC#N)CC3)c3c2cnc2ccsc23)c1]

### Step 1: 2-((1r,4r)-4-((6-(4-hydroxybut-1-yn-1-yl)thieno[3,2-b]pyridin-7-yl)amino)cyclohexyl)acetonitrile

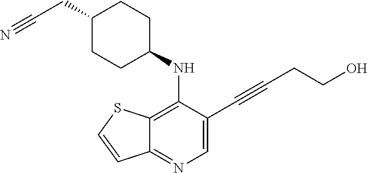 [SMILES: N#CC[C@H]1CC[C@H](Nc2c(C#CCCO)cnc3ccsc23)CC1]

To a screw-cap via equipped with a magnetic stir bar was added 2-((1r,4r)-4-((6-iodothieno[3,2-b]pyridin-7-yl)amino)cyclohexyl)acetonitrile (see step 2 in example 1, 263.3 mg, 0.663 mmol), Copper(I) iodide (76.6 mg, 0.402 mmol) and Bis(triphenylphosphine)palladium(II) dichloride (140.3 mg, 0.200 mmol). The vial was sealed with a Teflon-lined septum, evacuated and backfilled with nitrogen (this process was repeated a total of three times). A solution of but-3-yn-1-ol (93.2 mg, 1.330 mmol) in DMF (6.0 mL) was added via syringe, followed by triethylamine (335.6 mg, 3.32 mmol). The mixture was stirred at 60° C. for 16 h. After cooling to room temperature, the reaction was concentrated. The residue was purified on silica gel (20 g, 0-5% MeOH in CH₂Cl₂) to give the desired product (142.0 mg, 63%). LC-MS calculated for C₁₉H₂₂N₃OS (M+H)⁺: m/z=340.1; found: 340.1.

### Step 2: 2-((1r,4r)-4-(7-(2-hydroxyethyl)-8H-pyrrolo[2,3-d]thieno[3,2-b]pyridin-8-yl)cyclohexyl)acetonitrile

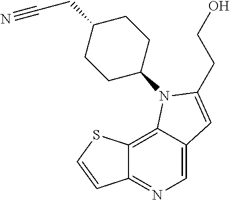 [SMILES: N#CC[C@H]1CC[C@H](n2c(CCO)cc3cnc4ccsc4c32)CC1]

To 2-((1r,4r)-4-((6-(4-hydroxybut-1-yn-1-yl)thieno[3,2-b]pyridin-7-yl)amino)cyclohexyl)acetonitrile (90.0 mg, 0.265 mmol) was added a solution of tetrabutylammonium fluoride (TBAF) (1.0 M in THF) (3.0 mL, 3.00 mmol). The mixture was stirred at 80° C. for 16 h. After cooling to room temperature, the mixture was purified using prep-LCMS (XBridge C18 column, eluting with a gradient of acetonitrile/water containing 0.15% NH₄OH, at flow rate of 60 mL/min) to afford the desired product (30.4 mg, 34%). LCMS calculated for C₁₉H₂₂N₃OS (M+H)⁺: m/z=340.1; found: 340.1.

### Step 3: 2-((1r,4r)-4-(6-bromo-7-(2-hydroxyethyl)-8H-pyrrolo[2,3-d]thieno[3,2-b]pyridin-8-yl)cyclohexyl)acetonitrile

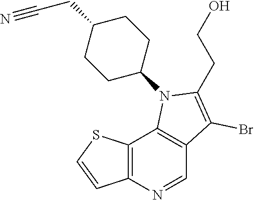 [SMILES: N#CC[C@H]1CC[C@H](n2c(CCO)c(Br)c3cnc4ccsc4c32)CC1]

To a mixture of 2-((1r,4r)-4-(7-(2-hydroxyethyl)-8H-pyrrolo[2,3-d]thieno[3,2-b]pyridin-8-yl)cyclohexyl)acetonitrile (30.4 mg, 0.090 mmol) and 1-bromopyrrolidine-2,5-dione (21.0 mg, 0.118 mmol) was added CH₂Cl₂ (2.0 mL) followed by DMF (2.0 mL). The mixture was stirred at room temperature for 2 h. The reaction mixture was concentrated. The residue was purified on silica gel (10 g, 0-100% EtOAc in CH₂Cl₂) to give the desired product as a white solid (23.9 mg, 64%). LC-MS calculated for C₁₉H₂₁BrN₃OS (M+H)⁺ m/z=418.1; found 418.0.

### Step 4: 3-(8-((1r,4r)-4-(cyanomethyl)cyclohexyl)-7-(2-hydroxyethyl)-8H-pyrrolo[2,3-d]thieno[3,2-b]pyridin-6-yl)-N,N-dimethylbenzamide

To a screw-cap vial equipped with a magnetic stir bar was added 2-((1r,4r)-4-(6-bromo-7-(2-hydroxyethyl)-8H-pyrrolo[2,3-d]thieno[3,2-b]pyridin-8-yl)cyclohexyl)acetonitrile (23.9 mg, 0.057 mmol), N,N-dimethyl-3-(4,4,5,5-tetramethyl-1,3,2-dioxaborolan-2-yl)benzamide (32.6 mg, 0.118 mmol), dicyclohexyl(2′,4′,6′-triisopropylbiphenyl-2-yl)phosphine-(2′-aminobiphenyl-2-yl)(chloro)palladium (1:1) (8.6 mg, 10.93 μmol) and cesium carbonate (74.9 mg, 0.230 mmol). The vial was sealed with a Teflon-lined septum, evacuated and backfilled with nitrogen (this process was repeated a total of three times). 1,4-Dioxane (3.0 mL) was added via syringe, followed by degassed water (300.0 μL). After stirring at 80° C. for 16 h, the reaction was cooled to room temperature. MeOH (2.0 mL) was added followed by HCl (4.0 M in dioxane) (1.0 mL, 4.00 mmol). After stirring at room temperature for 30 min, the reaction mixture was filtered. The filtrate was purified using prep-LCMS (XBridge C18 column, eluting with a gradient of acetonitrile/water containing 0.1% TFA, at flow rate of 60 mL/min) to afford the desired product as its TFA salt. LC-MS calculated for C₂₈H₃₁N₄O₂S (M+H)⁺: m/z=487.2; found: 487.2.

### Example A. In Vitro JAK Kinase Assay

The inhibition potency of the compounds was determined in JAK1, JAK2, JAK3 and TYK2 enzyme activity assays that measure peptide phosphorylation using FRET to detect product formation. Compounds were serially diluted in DMSO and 0.1 ul was transferred to the wells of black, regular volume 384-well plate by ECHO liquid handler (Labcyte, San Jose, CA) in duplicate.

For all kinase assays a 10 ul volume of enzyme in assay buffer (50 mM Tris, pH=7.5, 10 mM MgCl₂, 0.01% Brij-35, 0.1% BSA, 1 mM EGTA, 5% Glycerol and 5 mM DTT) was added to the plate. Reaction was started by addition of 10 ul assay buffer containing biotinylated peptide substrate (Biot-EQEDEPEGDYFEWLE-amide, Sigma-Aldrich) and ATP (final concentrations 500 nM and 1 mM respectively). Final concentrations of enzymes were as follows: JAK-1 (In-house, see ref. Blood. 2010 Apr. 15; (15) 3109)—2.73 nM, JAK-2 (BPS, Cat. #40450)—0.025 nM, JAK-3 (Carna, Cat. #08-046)—0.01 nM, TYK-2 (Invitrogen, Cat. #PR8440C)—1.75 nM.

The plate was incubated at 25° C. for 1 hr. The reactions were ended with the addition of 10 ul of quench solution (final concentrations: 50 mM Tris, pH=7.8, 150 mM NaCl, 0.5 mg/ml BSA, 15 mM EDTA, 48 nM APC/75 nM Streptavidin (Perkin-Elmer Cat. #CR130-100) and 1.5 nM Eu-antibody PY-20 (Perkin-Elmer Cat. #AD0067). After 20 min. incubation at RT plate was read on PHERAstar FS plate reader (BMG Labtech). Wells with no compound (only DMSO) served as the high signal controls, and those without enzyme were used as low signal controls. Half-maximal inhibitory concentration (IC50) determination was performed using the four parameter equation with Genedata software.

TABLE A

| Example | JAK1 IC₅₀ | JAK2/ | JAK3/ | TYK2 IC₅₀ | JAK2/ | JAK3/ |
|---|---|---|---|---|---|---|
| # | (nM) | JAK1 | JAK1 | (nM) | TYK2 | TYK2 |
| 1 | + | ≥10 | ≥10 | + | ≥10 | ≥10 |
| 2 | + | ≥10 | ≥10 | + | ≥4 | ≥10 |
| 3 | +++ | ≥4 | ≥10 | +++ | | ≥10 |
| 4 | ++ | ≥4 | ≥10 | +++ | | ≥10 |
| 5 | + | ≥10 | ≥10 | + | ≥10 | ≥10 |
| 6 | + | ≥4 | ≥10 | + | | ≥10 |
| 7 | +++ | | | ++ | | ≥4 |
| 8 | + | ≥4 | ≥10 | + | ≥4 | ≥10 |
| 9 | + | ≥10 | ≥10 | + | ≥4 | ≥10 |
| 10 | +++ | ≥4 | ≥10 | +++ | ≥4 | ≥10 |
| 11 | + | ≥4 | ≥10 | + | | ≥10 |
| 12 | +++ | ≥10 | ≥10 | ++++ | ≥4 | ≥10 |
| 13 | +++ | ≥10 | ≥10 | ++++ | | ≥10 |
| 14 | + | | ≥10 | + | | ≥10 |
| 15 | + | | ≥10 | + | | ≥10 |
| 16 | ++ | | ≥10 | ++ | | ≥10 |
| 17 | + | | ≥10 | ++ | | ≥10 |
| 18 | ++ | ≥4 | ≥10 | ++ | ≥4 | ≥10 |
| 19 | + | ≥4 | ≥10 | + | ≥4 | ≥10 |
| 20 | +++ | ≥4 | ≥10 | +++ | | ≥10 |
| 21 | ++ | | ≥10 | ++ | | ≥10 |
| 22 | + | ≥10 | ≥10 | + | | ≥10 |
| 23 | + | ≥10 | ≥10 | + | ≥4 | ≥10 |
| 24 | + | ≥4 | ≥10 | + | ≥4 | ≥10 |
| 25 | + | ≥10 | ≥10 | + | ≥4 | ≥10 |
| 26 | + | ≥10 | ≥10 | + | ≥10 | ≥10 |
| 27 | + | ≥10 | ≥10 | + | ≥10 | ≥10 |
| 28 | + | ≥10 | ≥10 | + | ≥10 | ≥10 |
| 29 | + | ≥10 | ≥10 | + | ≥10 | ≥10 |

In the table above, + means <50 nM, ++ means 50 nM to <100 nM, +++ means 100 to <300 nM, ++++ means 300-650 nM.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 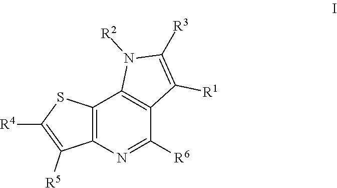
or a pharmaceutically acceptable salt thereof, wherein:
R¹ is -(L¹)-R^{X} or —R^{Y};
L¹ is C₁₋₆ alkylene, C₂₋₆ alkenylene, C₂₋₆ alkynylene, C₁₋₆ haloalkylene, —O—(C₁₋₆ alkylene)-, or —N(R^{L})—, wherein said C₁₋₆ alkylene, C₂₋₆ alkenylene, C₂₋₆ alkynylene, C₁₋₆ haloalkylene, and —O—(C₁₋₆ alkylene)- are each optionally substituted by 1, 2, 3, or 4 independently selected R^{G} substituents;
R^{L} is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
R^{X} is selected from Cy¹, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}NR^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1};
R^{Y} is selected from Cy¹, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k};
Cy¹ is C₃₋₁₀ cycloalkyl, 6-10 membered aryl, 4-10 membered heterocycloalkyl, or 5-10 membered heteroaryl, each of which is optionally substituted with 1, 2, 3, 4, 5, or 6 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂-6 alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{c1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each Rⁱ¹ and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b1}, NR^{c11}S(O)₂R^{b1}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or two R^{1A} substituents attached to C₃₋₁₀ cycloalkyl, 6-10 membered aryl, 4-10 membered heterocycloalkyl, or 5-10 membered heteroaryl, together with the atoms to which they are connected, form a C₃₋₇ cycloalkyl, phenyl, 4-7 membered heterocycloalkyl, or 5-6 membered heteroaryl ring, which is optionally substituted by 1, 2, 3, or 4 independently selected R^{Cy1} substituents;
each R^{Cy1} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b1}, NR^{c11}S(O)₂R^{b1}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
or, any R^{c12} and R^{d12} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{1C} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₄ alkyl, and 5-6 membered heteroaryl-C₁₋₄ alkyl, which are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1C} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, NHOR^{a13}, C(O)R^{b13}, C(O)NR^{c13}R^{d13}, C(O)NR^{c13}(OR^{a3}), C(O)OR^{a1}, OC(O)R^{b13}, OC(O)NR^{c13}R^{d13}, NR^{c13}R^{d13}, NR^{c13}NR^{c13}R^{d13}, NR^{c13}C(O)R^{b13}, NR^{c13}C(O)OR^{a13}, NR^{c13}C(O)NR^{c13}R^{d13}, C(═NR^{e13})R^{b13}, C(═NR^{e13})NR^{c13}R^{d13}, NR^{c13}C(═NR^{e13})NR^{c13}R^{d13}, NR^{c13}C(═NR^{e13})R^{b13}, NR^{c13}S(O)NR^{c13}R^{d13}, NR^{c13}S(O)R^{b13}, NR^{c13}S(O)₂R^{b13}, NR^{c13}S(O)(═NR^{e13})R^{b13}, NR^{c13}S(O)₂NR^{c13}R^{d13}, S(O)R^{b13}, S(O)NR^{c13}R^{d13}, S(O)₂R^{b13}, S(O)₂NR^{c13}R^{d13}, OS(O)(═NR^{e13})R^{b13}, OS(O)₂R^{b13}, S(O)(═NR^{e13})R^{b13}, SF₅, P(O)R^{f13}R^{g1}, OP(O)(OR^{h13})(ORⁱ¹³), P(O)(OR^{h13})(ORⁱ¹³), and BR^{j13}R^{k13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13}, and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c13} and R^{d13} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{G} substituents;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f13} and R^{g13} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h13} and Rⁱ¹³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j13} and R^{k13} is independently selected from OH, C₁₋₆ alkoxy, and C₁₋₆ haloalkoxy;
or any R^{j13} and R^{k13} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from C₁₋₆ alkyl, C₁₋₆ haloalkyl, C₂₋₆ alkenyl, C₂₋₆ alkynyl, Cy², and —(C₁₋₄ alkylene)-Cy², wherein said C₁₋₆ alkyl, C₁₋₆ haloalkyl, C₂₋₆ alkenyl, and C₂₋₆ alkynyl are each optionally substituted with 1, 2, 3, 4, 5, or 6 independently selected R^{2A} substituents;
Cy² is C₃₋₁₀ cycloalkyl, 4-10 membered heterocycloalkyl, 6-10 membered aryl, or 5-10 membered heteroaryl, each of which is optionally substituted with 1, 2, 3, 4, 5, or 6 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂-6 alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{2A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c2}C(O)R^{b21}, NR^{c21}C(O)OR^{a21}, NR^{c21}C(O)NR^{c21}R^{d21}, C(═NR^{e21})R^{b21}, C(═NR^{e21})R^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents;
or two R^{2A} substituents attached to C₃₋₁₀ cycloalkyl, 6-10 membered aryl, 4-10 membered heterocycloalkyl, or 5-10 membered heteroaryl, together with the atoms to which they are connected, form a C₃₋₇ cycloalkyl, phenyl, 4-7 membered heterocycloalkyl, or 5-6 membered heteroaryl ring, which is optionally substituted by 1, 2, 3, or 4 independently selected R^{Cy2} substituents;
each R^{Cy2} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})R^{c21}R^{d21}, NR^{c21}C(═NR^{e21})R^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 5- or 6-membered heteroaryl or a 4-10 membered heterocycloalkyl group, wherein the 5- or 6-membered heteroaryl or 4-10 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{e22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2C} substituents;
or, any R^{c22} and R^{d22} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{2C} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2C} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2C} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a23}, SR^{a23}, NHOR^{a23}, C(O)R^{b23}, C(O)NR^{c23}R^{d23}, C(O)NR^{c23}(OR^{a23}), C(O)OR^{a23}, OC(O)R^{b23}, OC(O)NR^{c23}R^{d23}, NR^{c23}R^{d23}, NR^{c23}NR^{c23}R^{d23}, NR^{c23}C(O)R^{b23}, NR^{c23}C(O)OR^{a23}, NR^{c23}C(O)NR^{c23}R^{d23}, C(═NR^{e23})R^{b23}, C(═NR^{e23})NR^{c23}R^{d23}, NR^{c23}C(═NR^{e23})NR^{c23}R^{d23}, NR^{c23}C(═NR^{e23})R^{b23}, NR^{c23}S(O)NR^{c23}R^{d23}, NR^{c23}S(O)R^{b23}, NR^{c23}S(O)₂R^{b23}, NR^{c23}S(O)(═NR^{e23})R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, OS(O)(═NR^{e23})R^{b23}, OS(O)₂R^{b23}, S(O)(═NR^{e23})R^{b23}, SF₅, P(O)R^{f23}R^{g23}, OP(O)(OR^{h23})(ORⁱ²³), P(O)(OR^{h23})(ORⁱ²³), and BR^{j23}R^{k23}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a23}, R^{c23}, and R^{d23} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c23} and R^{d23} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{G} substituents;
each R^{b23} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f23} and R^{g23} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h23} and Rⁱ²³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j23} and R^{k23} is independently selected from OH, C₁₋₆ alkoxy, and C₁₋₆ haloalkoxy;
or any R^{j23} and R^{k23} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, OR^{a3}, SR^{a3}, NHOR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{c3})R^{b3}, C(═NR^{e3})NR^{c3}R^{d3}, NR^{c3}C(═NR^{c3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, S(O)(═NR^{e3})R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3A} substituents;
or, any R^{c3} and R^{d3} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{3A} substituents;
each R^{b3} is independently selected from C₁₋₆ alkyl, C₁₋₆ haloalkyl, C₂₋₆ alkenyl, C₂-6 alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃ C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j3} and R^{k3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a31}SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}C(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c3}C(═NR^{e31})R^{b31}, NR^{c31}S(O)NR^{c31}R^{d31}, NR^{c31}S(O)R^{b31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, S(O)(═NR^{e31})R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, and C₃₋₄ cycloalkyl, wherein said C₁₋₆ alkyl, C₁₋₆ haloalkyl, C₂₋₆ alkenyl, C₂₋₆ alkynyl, and C₃₋₄ cycloalkyl are each optionally substituted with 1, 2, 3, or 4 independently selected R^{G} substituents;
or, any R^{c31} and R^{d31} attached to the same N atom, together with the N atom to which they are attached, form a 5- or 6-membered heteroaryl or a 4-7 membered heterocycloalkyl group, wherein the 5- or 6-membered heteroaryl or 4-7 membered heterocycloalkyl group is optionally substituted with 1, 2, 3, or 4 independently selected R^{G} substituents;
each R^{b31} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f31} and R^{g31} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h31} and Rⁱ³¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j31} and R^{k31} is independently selected from OH, C₁₋₆ alkoxy, and C₁₋₆ haloalkoxy;
or any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁴ is selected from H, halo, cyano, hydroxy, amino, (C₁₋₄ alkyl)amino, di(C₁₋₄ alkyl)amino, C₁₋₄ alkyl, C₁₋₄ haloalkyl, C₁₋₄ alkoxy, and C₁₋₄ haloalkoxy;
R⁵ is selected from H, halo, cyano, hydroxy, amino, (C₁₋₄ alkyl)amino, di(C₁₋₄ alkyl)amino, C₁₋₄ alkyl, C₁₋₄ haloalkyl, C₁₋₄ alkoxy, and C₁₋₄ haloalkoxy;
R⁶ is selected from H, halo, cyano, hydroxy, amino, (C₁₋₄ alkyl)amino, di(C₁₋₄ alkyl)amino, C₁₋₄ alkyl, C₁₋₄ haloalkyl, C₁₋₄ alkoxy, and C₁₋₄ haloalkoxy; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein R¹ is —R^{Y}.

3. The compound of claim 1, or a pharmaceutically acceptable salt thereof, wherein R^{Y} is Cy¹.

4. The compound of claim 1, or a pharmaceutically acceptable salt thereof, wherein Cy¹ is 6-10 membered aryl, 5-10 membered heteroaryl, or 6-10 membered heterocycloalkyl each of which is optionally substituted with 1, 2, 3, 4, 5, or 6 independently selected R^{1A} substituents.

5. The compound of claim 1, or a pharmaceutically acceptable salt thereof, wherein Cy¹ is phenyl, 5-6 membered heteroaryl, or 4-10 membered heterocycloalkyl each of which is optionally substituted with 1, 2, 3, or 4 independently selected R^{1A} substituents.

6. The compound of claim 1, or a pharmaceutically acceptable salt thereof, wherein Cy¹ is phenyl, which is optionally substituted with 1, 2, 3, or 4 independently selected R^{1A} substituents.

7. The compound of claim 1, or a pharmaceutically acceptable salt thereof, wherein Cy¹ is 5-6 membered heteroaryl, which is optionally substituted with 1, 2, 3, or 4 independently selected R^{1A} substituents.

8. The compound of claim 1, or a pharmaceutically acceptable salt thereof, wherein Cy¹ is 4-10 membered heterocycloalkyl, which is optionally substituted with 1, 2, 3, or 4 independently selected R^{1A} substituents.

9. The compound of claim 1, or a pharmaceutically acceptable salt thereof, wherein each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b1}, OC(O)NR^{c11}R^{d11}, NR^{c11}R^{d11}, NR^{c11}C(O)R^{b11}, NR^{c11}C(O)OR^{a1}, NR^{c11}C(O)NR^{c11}R^{d11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents.

10. The compound of claim 1, or a pharmaceutically acceptable salt thereof, wherein each R^{1A} is independently selected from halo, CN, NO₂, C₁₋₆ alkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b11}, C(O)NR^{c11}R^{d11}, and NR^{c11}C(O)R^{b11}, wherein said C₁₋₆ alkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents.

11. The compound of claim 1, or a pharmaceutically acceptable salt thereof, wherein each R^{1A} is independently selected from halo, C₃₋₁₀ cycloalkyl, 4-10 membered heterocycloalkyl, 4-10 membered heterocycloalkyl-C₁₋₄alkyl, C(O)R^{b11}, C(O)NR^{c11}R^{d11}, and NR^{c11}C(O)R^{b11}, wherein said C₃₋₁₀ cycloalkyl, 4-10 membered heterocycloalkyl, and 4-10 membered heterocycloalkyl-C₁₋₄ alkyl are each optionally substituted with 1, 2, 3, or 4 independently selected R^{1B} substituents.

12. The compound of claim 1, or a pharmaceutically acceptable salt thereof, wherein: each R^{a11}, R^{c11}, and R^{d11} is independently selected from H, C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-10 membered heterocycloalkyl group, which is optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12} and R^{d12} is independently selected from H, C₁₋₆ alkyl, and C₁₋₆ haloalkyl; and
each R^{b12} is independently selected from C₁₋₆ alkyl and C₁₋₆ haloalkyl.

13. The compound of claim 1, or a pharmaceutically acceptable salt thereof, wherein:
each R^{a11}, R^{c11}, and R^{d11} is independently selected from H, C₁₋₆ alkyl, and 4-7 membered heterocycloalkyl, wherein said C₁₋₆ alkyl and 4-7 membered heterocycloalkyl are each optionally substituted with 1, 2, or 3 independently selected R^{1B} substituents;
or, any R^{c11} and R^{d11} attached to the same N atom, together with the N atom to which they are attached, form a 4-10 membered heterocycloalkyl group, which is optionally substituted with 1, 2, or 3 independently selected R^{1B} substituents;
each R^{b11} is independently C₁₋₆ alkyl, which is optionally substituted with 1, 2, 3, or 4 independently selected R^{1B} substituents;
each R^{1B} is independently C₁₋₆ alkyl, OR^{a12}, and NR^{c12}R^{d12}; and
each R^{a12}, R^{c12}, and R^{d12} is independently selected from H and C₁₋₆ alkyl.

14. The compound of claim 1, or a pharmaceutically acceptable salt thereof, wherein R² is Cy².

15. The compound of claim 1, or a pharmaceutically acceptable salt thereof, wherein Cy² is C₃₋₁₀ cycloalkyl or 4-10 membered heterocycloalkyl, each of which is optionally substituted with 1, 2, 3, or 4 independently selected R^{2A} substituents.

16. The compound of claim 1, or a pharmaceutically acceptable salt thereof, wherein Cy² is C₃₋₇ cycloalkyl, which is optionally substituted with 1 or 2 independently selected R^{2A} substituents.

17. The compound of claim 1, or a pharmaceutically acceptable salt thereof, wherein Cy² is cyclohexyl, which is optionally substituted with 1 R^{2A} substituent.

18. The compound of claim 1, or a pharmaceutically acceptable salt thereof, wherein each R^{2A} is C₁₋₆ alkyl, which is optionally substituted with 1 or 2 independently selected R^{2B} substituents.

19. The compound of claim 1, or a pharmaceutically acceptable salt thereof, wherein:
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

20. The compound of claim 1, or a pharmaceutically acceptable salt thereof, wherein Cy² is cyclohexyl, which is substituted with —CH₂CN.

21. The compound of claim 1, or a pharmaceutically acceptable salt thereof, wherein R³ is selected from H, D, halo, CN, C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl, OR^{a3}, SR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl are each optionally substituted by 1, 2, 3, or 4 independently selected R^{3A} substituents.

22. The compound of claim 1, or a pharmaceutically acceptable salt thereof, wherein R³ is selected from C₁₋₆ alkyl and C₁₋₆ haloalkyl, which are each optionally substituted by 1 or 2 independently selected R^{3A} substituents.

23. The compound of claim 1, or a pharmaceutically acceptable salt thereof, wherein each R^{3A} is independently selected from halo, CN, OR^{a31}, and NR^{c31}R^{d31}, wherein each R^{a31}, R^{c31}, and R^{d31} is independently selected from H, C₁₋₆ alkyl, and C₁₋₆ haloalkyl.

24. The compound of claim 1, or a pharmaceutically acceptable salt thereof, wherein R⁴ is selected from H, halo, CN, hydroxy, amino, C₁₋₃ alkyl, and C₁₋₃ haloalkyl.

25. The compound of claim 1, or a pharmaceutically acceptable salt thereof, wherein R⁴ is selected from H, halo, and CN.

26. The compound of claim 1, or a pharmaceutically acceptable salt thereof, wherein R⁴ is H.

27. The compound of claim 1, or a pharmaceutically acceptable salt thereof, wherein R⁵ is selected from H, halo, CN, hydroxy, amino, C₁₋₃ alkyl, and C₁₋₃ haloalkyl.

28. The compound of claim 1, or a pharmaceutically acceptable salt thereof, wherein R⁵ is selected from H, halo, and CN.

29. The compound of claim 1, or a pharmaceutically acceptable salt thereof, wherein R⁵ is H.

30. The compound of claim 1, or a pharmaceutically acceptable salt thereof, wherein R⁶ is selected from H, halo, CN, hydroxy, amino, C₁₋₃ alkyl, and C₁₋₃ haloalkyl.

31. The compound of claim 1, or a pharmaceutically acceptable salt thereof, wherein R⁶ is selected from H, halo, and CN.

32. The compound of claim 1, or a pharmaceutically acceptable salt thereof, wherein R⁶ is H.

33. The compound of claim 1, or a pharmaceutically acceptable salt thereof, wherein R⁴, R⁵, and R⁶ are each H.

34. The compound of claim 1, or a pharmaceutically acceptable salt thereof, wherein:
R¹ is —R^{Y};
R is Cy¹;
Cy¹ is phenyl, 5-6 membered heteroaryl, or 4-10 membered heterocycloalkyl each of which is optionally substituted with 1, 2, 3, or 4 independently selected R^{1A} substituents;
each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b1}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d111}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-10 membered heterocycloalkyl group, which is optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, 4-10 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12} and R^{d12} is independently selected from H, C₁₋₆ alkyl, and C₁₋₆ haloalkyl;
each R^{b12} is independently selected from C₁₋₆ alkyl and C₁₋₆ haloalkyl;
R² is Cy²;
Cy² is C₃₋₁₀ cycloalkyl or 4-10 membered heterocycloalkyl, each of which is optionally substituted with 1, 2, 3, or 4 independently selected R^{2A} substituents;
each R^{2A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R³ is selected from H, D, halo, CN, C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl, OR^{a3}, SR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, C₂₋₆ alkenyl, C₂₋₆ alkynyl, C₃₋₆ cycloalkyl, phenyl, 4-6 membered heterocycloalkyl, 5-6 membered heteroaryl, C₃₋₆ cycloalkyl-C₁₋₄ alkyl, phenyl-C₁₋₄ alkyl, 4-6 membered heterocycloalkyl-C₁₋₄ alkyl, and 5-6 membered heteroaryl-C₁₋₄ alkyl are each optionally substituted by 1, 2, 3, or 4 independently selected R^{3A} substituents;
each R^{3A} is independently selected from halo, CN, OR^{a31}, and NR^{c31}R^{d31}, wherein each R^{a31}, R^{c31}, and R^{d31} is independently selected from H, C₁₋₆ alkyl, and C₁₋₆ haloalkyl;
each R^{a31}, R^{c31}, and R^{d31} is independently selected from H, C₁₋₆ alkyl, and C₁₋₆ haloalkyl;
R⁴ is selected from H, halo, CN, hydroxy, amino, C₁₋₃ alkyl, and C₁₋₃ haloalkyl;
R⁵ is selected from H, halo, CN, hydroxy, amino, C₁₋₃ alkyl, and C₁₋₃ haloalkyl; and
R⁶ is selected from H, halo, CN, hydroxy, amino, C₁₋₃ alkyl, and C₁₋₃ haloalkyl.

35. The compound of claim 1, or a pharmaceutically acceptable salt thereof, wherein:
R¹ is —R^{Y};
R is Cy¹;
Cy¹ is phenyl, 5-6 membered heteroaryl, or 4-10 membered heterocycloalkyl each of which is optionally substituted with 1, 2, 3, or 4 independently selected R^{1A} substituents;
each R^{a11}, R^{c11}, and R^{d11} is independently selected from H, C₁₋₆ alkyl, and 4-7 membered heterocycloalkyl, wherein said C₁₋₆ alkyl and 4-7 membered heterocycloalkyl are each optionally substituted with 1, 2, or 3 independently selected R^{1B} substituents;
or, any R^{c11} and R^{d11} attached to the same N atom, together with the N atom to which they are attached, form a 4-10 membered heterocycloalkyl group, which is optionally substituted with 1, 2, or 3 independently selected R^{1B} substituents;
each R^{b11} is independently C₁₋₆ alkyl, which is optionally substituted with 1, 2, 3, or 4 independently selected R^{1B} substituents;
each R^{1B} is independently C₁₋₆ alkyl, OR^{a12}, and NR^{c12}R^{d12};
each R^{a12}, R^{c12} and R^{d12} is independently selected from H and C₁₋₆ alkyl;
R² is Cy²;
Cy² is C₃₋₇ cycloalkyl, which is optionally substituted with 1 or 2 independently selected R^{2A} substituents;
each R^{2A} is C₁₋₆ alkyl, which is optionally substituted with 1 or 2 independently selected R^{2B} substituents;
each R^{a21}, R^{c21}, and R^{d21} is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{2B} substituents;
each R^{b21} is independently selected from C₁₋₆ alkyl and C₁₋₆ haloalkyl, which are each optionally substituted with 1 or 2 independently selected R^{2B} substituents;
each R^{2B} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R³ is selected from C₁₋₆ alkyl and C₁₋₆ haloalkyl, which are each optionally substituted by 1 or 2 independently selected R^{3A} substituents;
each R^{3A} is independently selected from halo, CN, OR^{a31}, and NR^{c31}R^{d31};
each R^{a31}, R^{c31}, and R^{d31} is independently selected from H, C₁₋₆ alkyl, and C₁₋₆ haloalkyl;
R⁴ is selected from H, halo, and CN;
R⁵ is selected from H, halo, and CN; and
R⁶ is selected from H, halo, and CN.

36. The compound of claim 1, wherein the compound or the salt is a compound of Formula II: 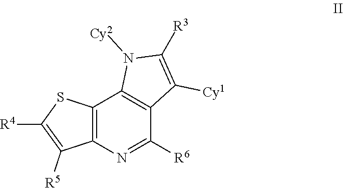
or a pharmaceutically acceptable salt thereof.

37. The compound of claim 1, wherein the compound or the salt is a compound of Formula IIa: 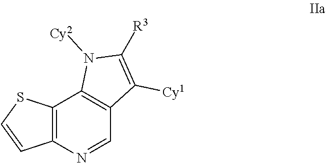
or a pharmaceutically acceptable salt thereof.

38. The compound of claim 1, wherein the compound or the salt is a compound of Formula IV: 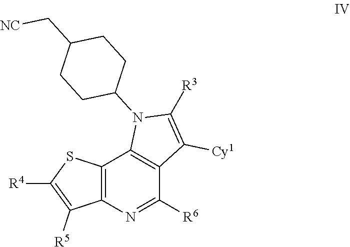
or a pharmaceutically acceptable salt thereof.

39. The compound of claim 1, wherein the compound or the salt is a compound of Formula IVa: 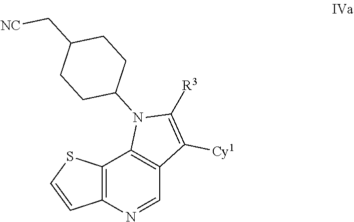
or a pharmaceutically acceptable salt thereof.

40. The compound of claim 1, which is selected from:
3-(8-((1r,4r)-4-(cyanomethyl)cyclohexyl)-7-methyl-8H-pyrrolo[2,3-d]thieno[3,2-b]pyridin-6-yl)-N,N-dimethylbenzamide;
2-((1r,4r)-4-(7-methyl-6-(3-(pyrrolidine-1-carbonyl)phenyl)-8H-pyrrolo[2,3-d]thieno[3,2-b]pyridin-8-yl)cyclohexyl)acetonitrile;
2-((1r,4r)-4-(7-methyl-6-(3-oxoisoindolin-5-yl)-8H-pyrrolo[2,3-d]thieno[3,2-b]pyridin-8-yl)cyclohexyl)acetonitrile;
N-(3-(8-((1r,4r)-4-(cyanomethyl)cyclohexyl)-7-methyl-8H-pyrrolo[2,3-d]thieno[3,2-b]pyridin-6-yl)phenyl)acetamide;
4-(8-((1r,4r)-4-(cyanomethyl)cyclohexyl)-7-methyl-8H-pyrrolo[2,3-d]thieno[3,2-b]pyridin-6-yl)-N-methylpicolinamide;
2-((1r,4r)-4-(7-methyl-6-(2-(4-methylpiperazin-1-yl)pyridin-4-yl)-8H-pyrrolo[2,3-d]thieno[3,2-b]pyridin-8-yl)cyclohexyl)acetonitrile;
2-((1r,4r)-4-(6-(4-fluoro-3-(pyrrolidin-1-ylmethyl)phenyl)-7-methyl-8H-pyrrolo[2,3-d]thieno[3,2-b]pyridin-8-yl)cyclohexyl)acetonitrile;
5-(8-((1r,4r)-4-(cyanomethyl)cyclohexyl)-7-methyl-8H-pyrrolo[2,3-d]thieno[3,2-b]pyridin-6-yl)-2-fluoro-N,N-dimethylbenzamide;
4-(8-((1r,4r)-4-(cyanomethyl)cyclohexyl)-7-methyl-8H-pyrrolo[2,3-d]thieno[3,2-b]pyridin-6-yl)-N,N-dimethylbenzamide;
4-(8-((1r,4r)-4-(cyanomethyl)cyclohexyl)-7-methyl-8H-pyrrolo[2,3-d]thieno[3,2-b]pyridin-6-yl)-3-fluoro-N,N-dimethylbenzamide;
4-(8-((1r,4r)-4-(cyanomethyl)cyclohexyl)-7-methyl-8H-pyrrolo[2,3-d]thieno[3,2-b]pyridin-6-yl)-2-fluoro-N,N-dimethylbenzamide;
5-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-2-carboxamide;
5-(8-((1r,4r)-4-(cyanomethyl)cyclohexyl)-7-methyl-8H-pyrrolo[2,3-d]thieno[3,2-b]pyridin-6-yl)pyrimidine-2-carbonitrile;
2-((1r,4r)-4-(6-(1-isopropyl-1H-pyrazol-4-yl)-7-methyl-8H-pyrrolo[2,3-d]thieno[3,2-b]pyridin-8-yl)cyclohexyl)acetonitrile;
2-((1r,4r)-4-(7-methyl-6-(1-(tetrahydro-2H-pyran-4-yl)-1H-pyrazol-4-yl)-8H-pyrrolo[2,3-d]thieno[3,2-b]pyridin-8-yl)cyclohexyl)acetonitrile;
2-((1r,4r)-4-(6-(4-(1-aminocyclopropyl)phenyl)-7-methyl-8H-pyrrolo[2,3-d]thieno[3,2-b]pyridin-8-yl)cyclohexyl)acetonitrile;
2-((1r,4r)-4-(6-(4-(1-aminocyclobutyl)phenyl)-7-methyl-8H-pyrrolo[2,3-d]thieno[3,2-b]pyridin-8-yl)cyclohexyl)acetonitrile;
2-((1r,4r)-4-(7-methyl-6-(pyrimidin-2-yl)-8H-pyrrolo[2,3-d]thieno[3,2-b]pyridin-8-yl)cyclohexyl)acetonitrile;
2-((1r,4r)-4-(7-methyl-6-(pyridazin-4-yl)-8H-pyrrolo[2,3-d]thieno[3,2-b]pyridin-8-yl)cyclohexyl)acetonitrile;
2-((1r,4r)-4-(7-methyl-6-(pyrazin-2-yl)-8H-pyrrolo[2,3-d]thieno[3,2-b]pyridin-8-yl)cyclohexyl)acetonitrile;
2-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-4-carboxamide;
2-(8-((1r,4r)-4-(cyanomethyl)cyclohexyl)-7-methyl-8H-pyrrolo[2,3-d]thieno[3,2-b]pyridin-6-yl)-N,N-dimethylpyrimidine-5-carboxamide;
2-((1r,4r)-4-(6-(3-(6-oxa-2-azaspiro[3.4]octane-2-carbonyl)phenyl)-7-methyl-8H-pyrrolo[2,3-d]thieno[3,2-b]pyridin-8-yl)cyclohexyl)acetonitrile;
2-((1r,4r)-4-(7-methyl-6-(3-(7-oxo-2,6-diazaspiro[3.4]octane-2-carbonyl)phenyl)-8H-pyrrolo[2,3-d]thieno[3,2-b]pyridin-8-yl)cyclohexyl)acetonitrile;
2-((1r,4r)-4-(6-(3-(3-hydroxy-3-methylazetidine-1-carbonyl)phenyl)-7-methyl-8H-pyrrolo[2,3-d]thieno[3,2-b]pyridin-8-yl)cyclohexyl)acetonitrile;
2-((1R,4r)-4-(6-(3-((R)-3-methoxypyrrolidine-1-carbonyl)phenyl)-7-methyl-8H-pyrrolo[2,3-d]thieno[3,2-b]pyridin-8-yl)cyclohexyl)acetonitrile;
2-((1r,4r)-4-(7-methyl-6-(3-(morpholine-4-carbonyl)phenyl)-8H-pyrrolo[2,3-d]thieno[3,2-b]pyridin-8-yl)cyclohexyl)acetonitrile;
2-((1r,4r)-4-(7-methyl-6-(3-(4-methylpiperazine-1-carbonyl)phenyl)-8H-pyrrolo[2,3-d]thieno[3,2-b]pyridin-8-yl)cyclohexyl)acetonitrile; and
3-(8-((1r,4r)-4-(cyanomethyl)cyclohexyl)-7-(2-hydroxyethyl)-8H-pyrrolo[2,3-d]thieno[3,2-b]pyridin-6-yl)-N,N-dimethylbenzamide;
or a pharmaceutically acceptable salt of any of the aforementioned.

41. A pharmaceutical composition, comprising a compound of claim 1, or a pharmaceutically acceptable salt thereof, and a pharmaceutically acceptable carrier.

42. A method of inhibiting JAK1 and/or TYK2 in a patient in need thereof, comprising contacting a compound of claim 1, or a pharmaceutically acceptable salt thereof, with JAK1 and/or TYK2.

43. A method of inhibiting JAK1 and/or TYK2 in a patient in need thereof, comprising administering to the patient a therapeutically effective amount of a compound claim 1, or a pharmaceutically acceptable salt thereof.

44. A method of inhibiting JAK1 and TYK2 in a patient in need thereof, comprising administering to the patient a therapeutically effective amount of a compound of claim 1, or a pharmaceutically acceptable salt thereof.

45. A method of treating a disease or disorder associated with JAK1 and/or TYK2 in a patient in need thereof, comprising administering to the patient a therapeutically effective amount of a compound of claim 1, or a pharmaceutically acceptable salt thereof.

46. The method of claim 45, wherein the administration is topical administration.

47. The method of claim 45, wherein the administration is oral administration.

48. The method of claim 45, wherein the disease or disorder is a skin disease or disorder.

49. The method of claim 48, wherein the skin disease or disorder is selected from eczema, atopic dermatitis, psoriasis, contact dermatitis, vitiligo, hidradenitis suppurativa, prurigo nodularis, ichthyosis, lichen planus, lichen sclerosus, alopecia, rosacea, urticarial, non-melanoma skin cancer, dermatomyositis, bullous pemphigoid, pemphigus vulgaris, cutaneous graft-versus-host disease, cutaneous manifestations of interferonopathies, STAT1 gain-of-function, STAT3 gain-of-function, systemic lupus erythematosus, cutaneous lupus, and scleroderma.

50. The method of claim 45, wherein the disease or disorder is an ophthalmic disease or disorder.

51. The method of claim 50, wherein the ophthalmic disease or disorder is selected from ocular graft-versus-host disease, dry eye, uveitis, scleritis, Behcet's disease, and Sjögren's syndrome.

52. The method of claim 45, wherein the disease or disorder is a respiratory disease or disorder.

53. The method of claim 52, wherein the respiratory disease or disorder is selected from asthma, chronic obstructive pulmonary disease, rhinitis, and idiopathic pulmonary fibrosis.

54. The method of claim 45, wherein the disease or disorder is selected from a rectal disease or disorder.

55. The method of claim 54, wherein the rectal disease or disorder is selected from ulcerative colitis, Crohn's disease, celiac disease, Peutz-Jeghers syndrome, juvenile polyposis syndrome, and Cowden Syndrome.

56. The method of claim 45, wherein the disease or disorder is cancer.

57. The method of claim 45, wherein disease or disorder is neuroinflammation.

58. The method of claim 57, wherein the neuroinflammation is amyotrophic lateral sclerosis (ALS), frontotemporal dementia (FTD), Alzheimer's disease, multiple sclerosis or Parkinson's disease.
